(19) United States
(12) Patent Application Publication
(10) Pub. No.: US 2023/0263247 A1
(43) Pub. Date: Aug. 24, 2023

(54) EXAMINATION GARMENT FOR ENDOSCOPY

(71) Applicant: FUJIFILM Corporation, Tokyo (JP)

(72) Inventors: Takumi DEJIMA, Kanagawa (JP); Tomohiro OHKI, Kanagawa (JP); Kenji NAGAMIYA, Kanagawa (JP); Shozo IYAMA, Kanagawa (JP)

(73) Assignee: FUJIFILM Corporation, Tokyo (JP)

(21) Appl. No.: 18/174,543

(22) Filed: Feb. 24, 2023

Related U.S. Application Data
(63) Continuation of application No. PCT/JP2021/031160, filed on Aug. 25, 2021.

(30) Foreign Application Priority Data
Aug. 31, 2020 (JP) 2020-145854

(51) Int. Cl. A41D 13/12

(52) U.S. Cl. CPC A41D 13/1254

(57) ABSTRACT

An examination garment for endoscopy includes a covering portion and a first fluid suppression member. A pants-type covering portion has an insertion port that covers a subject, who is a test object, from a torso portion to a thigh portion, and allows an insertion part of the endoscope to be inserted thereinto. The first fluid suppression member is provided in the insertion port of the covering portion and suppresses passage of the fluid in the insertion port.

### CROSS-REFERENCE TO RELATED APPLICATIONS

This application is a Continuation of PCT International Application No. PCT/JP2021/031160 filed on 25 Aug. 2021, which claims priority under 35 U.S.C §119(a) to Japanese Patent Application No. 2020-145854 filed on 31 Aug. 2020. The above application is hereby expressly incorporated by reference, in its entirety, into the present application.

### BACKGROUND OF THE INVENTION

### 1. Field of the Invention

The present invention relates to an examination garment for endoscopy, which is worn by a subject during an endoscopy.

### 2. Description of the Related Art

In the medical field, endoscopes for observing a lower gastrointestinal tract such as a large intestine are known in which an insertion part is inserted into the lower gastrointestinal tract to perform observation. In a case where observation is performed by the endoscopes for observing the lower gastrointestinal tract, a subject wears an examination garment.

In the related art, as examination garments for endoscopy, pants-type examination garments having a torso hole and two thigh holes are known. In such examination garments, an opening portion for inserting the insertion part of each endoscope is formed on a rear surface side. JP2014-237913A describes a diaper material, that is, a pants-type examination garment formed of an absorbent material. In this examination garment made of the diaper material, an inverted T-shaped cut end (opening portion) is provided on the rear surface side such that the insertion part of the endoscope can be inserted or removed, and the periphery of the cut end is reinforced by locking or overlocking. Additionally, in the examination garment made of the diaper material, a tape-like cloth having contractility and a preventive gathering are provided around the thigh hole such that the body fluid does not flow out from the thigh hole.

Meanwhile, in an endoscopy in which the lower gastrointestinal tract is observed, an exact situation is not ascertained in a case where there is excrement (stool) in the intestine. Therefore, as pretreatment, the endoscopy is often performed after a purgative and an intestinal cleanser are taken and excrement in the intestine is discharged.

### SUMMARY OF THE INVENTION

However, in the case of a subject who undergoes insufficient pretreatment or a subject who requires an emergency due to melena or the like, the endoscopy may have to be performed in a state in which the pretreatment is insufficient. In this case, body fluids such as the excrement in the intestine and blood are vigorously released together with air and washing water fed during the examination. Therefore, in the examination garment of the diaper material as described in the above JP2014-237913A, the body fluids released from the subject may exceed the capacity that can be absorbed by the examination garment. For this reason, a body fluid may leak from an opening portion for inserting the insertion part of the endoscope.

In the medical field, it is desired to take further preventive measures against infectious diseases. In particular, it is important to take measures to prevent infection caused by the contact of contents such as the body fluid of an infectious disease patient with mucous membranes of eyes, a mouth, or the like and infection caused by inhalation of droplets released from the infectious disease patient. Thus, it is required to prevent leakage of the body fluid as well as the droplets in the examination garment for endoscopy.

An object of the present invention is to provide an examination garment for endoscopy that can prevent leakage of a body fluid and droplets in a case where an insertion part of an endoscope is inserted through an anus of a subject.

An examination garment for endoscopy according to an aspect of the present invention comprises a covering portion and a first fluid suppression member. The covering portion covers a subject and has an insertion port into which an insertion part of an endoscope is inserted. A first fluid suppression member is provided at the insertion port to suppress passage of a fluid in the insertion port.

It is preferable that the covering portion has a suction tube port into which a suction tube is inserted, and it is more preferable that the covering portion includes a second fluid suppression member that is provided in the suction tube port and that suppresses the passage of the fluid in the suction tube port.

It is preferable that the covering portion is a pants-type covering portion including a torso hole through which a torso portion of a subject passes and a thigh hole through which a thigh portion passes.

It is preferable that the covering portion has a contractible member, which contracts the thigh hole, provided around the thigh hole. It is preferable that the contractible member is a string or an elastic member that is doubly wrapped around the thigh hole. Alternatively, it is preferable that the contractible member is a gathering that is disposed around the thigh hole.

It is preferable that the covering portion is formed of a planar sheet capable of being wrapped around the subject.

It is preferable that the sheet has a quadrangular outer shape in a case where the sheet is brought into an unfolded state, or has a T-shaped outer shape in a case where the sheet is brought into an unfolded state.

It is preferable to further comprise a holding member that holds a state in which the covering portion is wrapped around the subject.

It is preferable to further comprise a fixing member that attaches at least one of the first or second fluid suppression member to the covering portion.

It is preferable that at least one of the first or second fluid suppression member has at least a first porous member in which a first slit parallel to an insertion direction of the insertion part is formed, and has a second porous member in which a second slit parallel to the insertion direction and intersecting the first slit is formed.

It is preferable that at least one of the first or second fluid suppression member has a first porous member in which three first slits are formed and a second porous member in which three second slits are formed.

It is preferable that the first porous member and the second porous member have a rectangular outer shape, one of the first slits is disposed to be perpendicular to one side of the first porous member, one of the second slits is disposed to be perpendicular to one side of the second porous member, and the first porous member and the second porous member are attached to the covering portion in a state in which the second slit is disposed at a position rotated by 180° around a central axis parallel to the insertion direction with respect to the first slit.

It is preferable that at least one of the first or second fluid suppression member is attached to the covering portion in a state in which the first porous member and the second porous member are laminated.

It is preferable that at least one of the first or second fluid suppression member has a duckbill valve that maintains airtightness of a pipe line in a state in which the insertion part is not inserted, and a thin plate member that has a through-hole with an inner diameter that matches an outer diameter of the insertion part and maintains the airtightness of the pipe line in a state in which the insertion part is inserted, and is attached to the covering portion in a state in which the duckbill valve and the thin plate member are laminated.

It is preferable that the fixing member includes a tubular member that extends to an outside of the covering portion in a case where the covering portion has covered the subject. It is preferable that at least one of the first or second fluid suppression member has a convex portion that protrudes to the outside of the covering portion in a case where the covering portion has covered the subject.

It is preferable that the covering portion is formed of a transparent or translucent member.

It is preferable that an absorbent material that absorbs a liquid is provided inside the covering portion. It is preferable that the absorbent material is attachable and detachable with respect to the covering portion. It is preferable that the absorbent material is provided with a discolored portion of which color changes by absorbing the liquid.

It is preferable that in a case where the covering portion has covered a periphery of a torso hole or the subject, a gathering is provided at a point located on a torso portion of the subject. It is preferable that a side surface portion and a thigh portion of the covering portion are provided with a fragile portion that is separable with a user’s force.

It is preferable that the covering portion has a cleaning liquid injection port for injecting a cleaning liquid thereinto. It is preferable that the covering portion has a storage portion that stores a liquid.

According to the present invention, it is possible to prevent leakage of the body fluid and droplets when the insertion part of the endoscope is inserted through the anus of the subject.

### BRIEF DESCRIPTION OF THE DRAWINGS

FIG. 1 is a schematic view showing an endoscopy using an endoscope for observing a lower gastrointestinal tract and an examination garment.

FIG. 2 is a front perspective view of the examination garment for endoscopy.

FIG. 3 is a rear perspective view of the examination garment for endoscopy.

FIG. 4 is a rear view of the examination garment for endoscopy.

FIG. 5 is a cross-sectional view of major parts of the examination garment for endoscopy.

FIG. 6 is an exploded perspective view showing a configuration of the examination garment for endoscopy.

FIG. 7 is a perspective view showing a configuration of a first fluid suppression member.

FIG. 8 is an explanatory diagram illustrating a state in which an insertion part is inserted into the fluid suppression member.

FIG. 9 is an explanatory diagram showing a case where the first fluid suppression member is attached to a covering portion.

FIG. 10 is an explanatory diagram illustrating an operation in performing an endoscopy on a subject who wears the examination garment for endoscopy.

FIG. 11 is an explanatory diagram illustrating a state in which the insertion part and a suction tube are inserted into the inside of the covering portion.

FIG. 12 is a cross-sectional view of major parts showing an example in which a storage portion and an absorbent material are disposed in an overlapping manner inside the covering portion.

FIG. 13 is an explanatory diagram illustrating an operation in a case where an endoscopy is performed on the subject who wears the examination garment for endoscopy in a second embodiment.

FIG. 14 is a perspective view of a first fluid suppression member and a first fixing member in the second embodiment.

FIG. 15 is an exploded perspective view of the first fluid suppression member and the first fixing member in the second embodiment.

FIG. 16 is a perspective view of an examination garment for endoscopy of a first modification example.

FIG. 17 is a perspective view showing a state in which the examination garment for endoscopy of the first modification example is separated.

FIG. 18 is an exploded perspective view showing a configuration of an examination garment for endoscopy in a second modification example.

FIG. 19 is a perspective view showing a configuration of a fluid suppression member in the second modification example.

FIGS. 20A and 20B are perspective views showing a configuration of a fluid suppression member in a third modification example.

FIG. 21 is an exploded perspective view of a fluid suppression member and a first fixing member in the third modification example.

FIG. 22 is a cross-sectional view of major parts of an examination garment for endoscopy in the third modification example.

FIG. 23 is a perspective view of the subject that is wearing an examination garment for endoscopy in a third embodiment.

FIG. 24 is a perspective view of a state in which the examination garment for endoscopy in the third embodiment is unfolded.

FIG. 25 is an explanatory diagram illustrating a method of having the subject wear the examination garment for endoscopy in the third embodiment.

FIG. 26 is a perspective view of the subject that is wearing an examination garment for endoscopy in a fourth modification example.

FIG. 27 is a perspective view of a state in which the examination garment for endoscopy in a fifth modification example is unfolded.

FIG. 28 is an explanatory diagram illustrating a method of having the subject wear the examination garment for endoscopy in the fifth modification example and wrapping a vertical line portion of the covering portion around the subject.

FIG. 29 is an explanatory diagram illustrating a method of having the subject wear the examination garment for endoscopy in the fifth modification example and winding a horizontal line portion of the covering portion around the subject.

### DESCRIPTION OF THE PREFERRED EMBODIMENTS

### First Embodiment

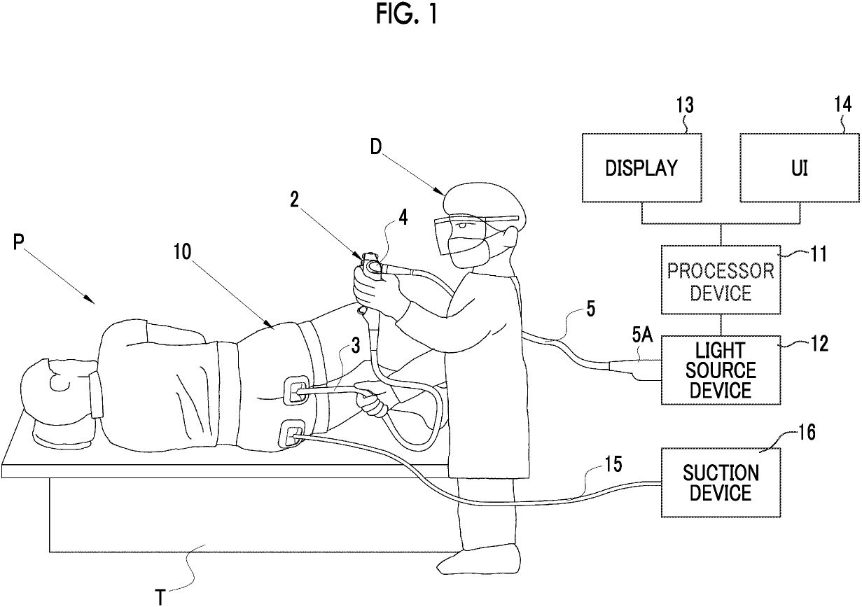

As shown in FIG. 1, an examination garment 10 for endoscopy (hereinafter, referred to as an examination garment 10) according to the present invention is used in an endoscopy for observing a lower gastrointestinal tract such as a large intestine. An endoscope 2 includes an insertion part 3, an operating part 4, and a universal cord 5. The insertion part 3 is inserted transanally into the lower gastrointestinal tract of a subject P who is a test object. The operating part 4 is consecutively installed in a proximal end part of the insertion part 3. The universal cord 5 is connected to the operating part 4. In addition, FIG. 1 shows a state in which a doctor D, who is a user, grips the operating part 4 with one hand and the insertion part 3 with the other hand, and inserts the insertion part 3 into a body of the subject P through the examination garment 10. Additionally, the subject P is undergoing the examination in a lateral decubitus posture (a state of lying sideways) on an examination table T.

The universal cord 5 is connected to an external device such as a processor device 11 or a light source device 12 via a connector 5A. The processor device 11 is electrically connected to a display 13 and to a user interface (UI) 14. The UI 14 includes a keyboard, a mouse, a touch pad, a microphone, and the like, and receives an input operation of the doctor D who is a user.

Although not shown, a distal end surface of the insertion part 3 is provided with an observation window and an illumination window. An image sensor (not shown) or the like is disposed behind the observation window, and an optical fiber cable (not shown) is disposed behind the illumination window. A signal line of the image sensor and the optical fiber cable are connected to the processor device 11 and to the light source device 12, respectively, through the insertion part 3, the operating part 4, and the universal cord 5. The processor device 11 performs image processing or the like on an endoscopic image captured by the image sensor and displays the image-processed image on the display 13.

Additionally, a suction tube 15 is inserted into the examination garment 10 in addition to the insertion part 3 of the endoscope 2. The suction tube 15 is connected to a suction device 16. The suction device 16 is operated by the operation of the doctor D or a helper to generate a negative pressure. By operating the suction device 16, a body fluid inside the examination garment 10 is suctioned through the suction tube 15. In addition, the suction device 16 may be a drain that performs suction via the manual operation of the doctor D or a helper or may be a suction pump that automatically generates a negative pressure.

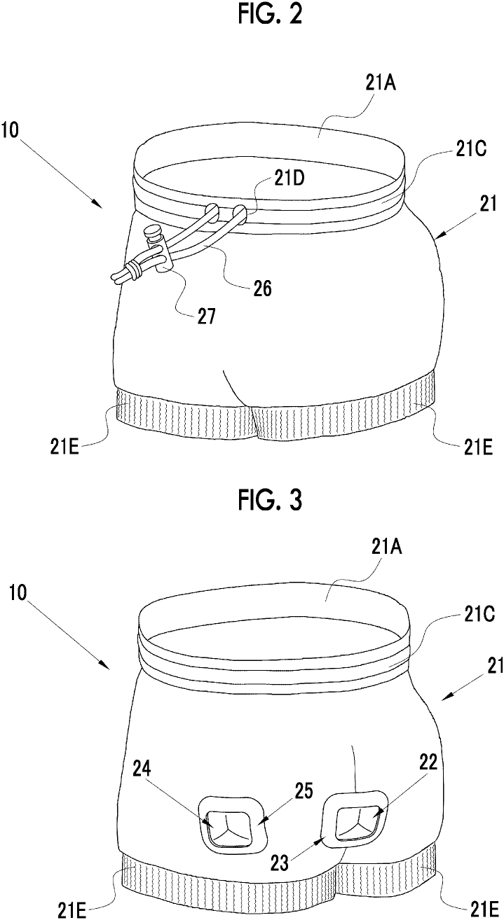

As shown in FIGS. 2 and 3, the examination garment 10 comprises a covering portion 21 worn by the subject P, a first fluid suppression member 22, a first fixing member 23, a second fluid suppression member 24, a second fixing member 25, a string 26, and a string fastener 27. In the present embodiment, the covering portion 21 is a pants-type covering portion that covers at least a part of the subject P from a torso portion to a thigh portion. The covering portion 21 is a transparent or translucent member, and is formed of, for example, a transparent or translucent vinyl sheet. The term “translucent” as used herein means that the interior can be visually recognized because the covering portion 21 is lightly colored but has transmittance. Additionally, on an upper body of the subject P that is not covered with the covering portion 21, an examination garment for the upper body may be worn separately from the examination garment 10.

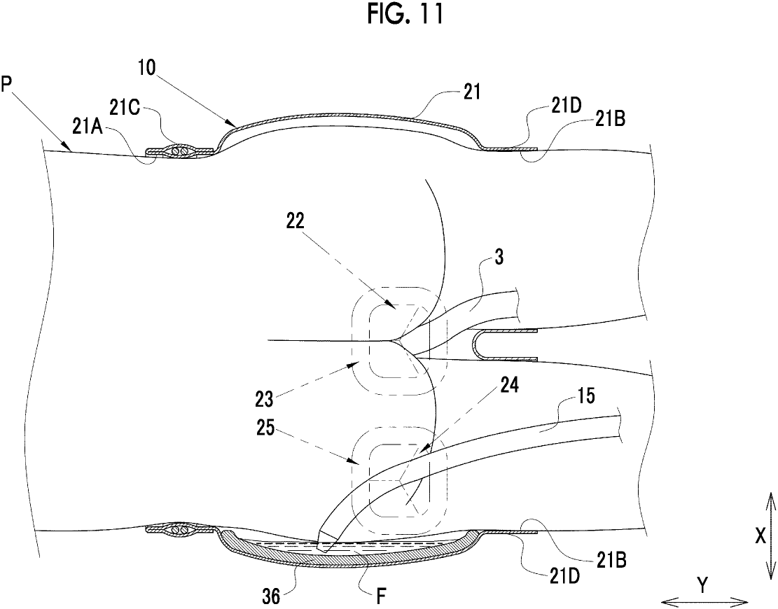

The covering portion 21 has a torso hole 21A and two thigh holes 21B (refer to FIG. 11). The torso hole 21A and the thigh hole 21B communicate with the inside of the covering portion 21. A string passing portion 21C is formed around the torso hole 21A. The string 26 is disposed inside the string passing portion 21C. Both end parts of the string 26 are exposed to the outside of the string passing portion 21C through an opening portion 21D of the string passing portion 21C. Both of the end parts of the string 26 are tied together by the string fastener 27. By changing the relative position of the string fastener 27 with respect to the string 26, the length of the string 26 can be adjusted in accordance with the size of the waist circumference of the subject P. Accordingly, the body fluid and the droplets are prevented from leaking from the torso hole 21A. In addition, it is preferable that the string 26 is doubly wrapped around the torso hole 21A. In addition, instead of the string 26, an elastic member such as rubber may be doubly wrapped around the torso hole 21A.

A gathering 21E serving as a contractible member is provided around the thigh hole 21B. The gathering 21E is disposed by, for example, sewing or knitting an elastic member (not shown) such as a contractible rubber. Accordingly, the thigh hole 21B is contractible. In a case where the subject P has passed the thigh portion through the thigh hole 21B, the thigh hole 21B is brought into close contact with the thigh portion due to the contraction of the gathering 21E. Accordingly, the body fluid and droplets can be prevented from leaking from the thigh hole 21B. In addition, the contractible member provided in the thigh hole 21B is not limited to the gathering 21E, and similarly to the torso hole 21A, a doubly-wrapped string or rubber may be provided around the thigh hole 21B, and the thigh hole 21B may be brought into close contact with the thigh portion. Additionally, the same gathering 21E as that of the thigh hole 21B may be provided as the contractible member provided in the torso hole 21A.

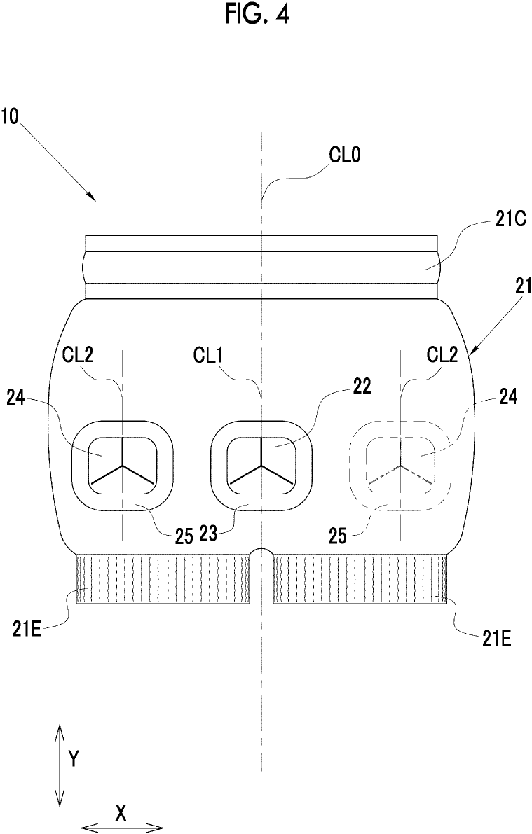

As shown in FIG. 4, the first fluid suppression member 22 is disposed at the center of the covering portion 21 on a rear surface side. Specifically, the first fluid suppression member 22 is disposed at a position where a center line CL0 of the covering portion 21 in a left-right direction X and a center line CL1 of the first fluid suppression member 22 in the left-right direction X coincide with each other. The first fluid suppression member 22 is attached to the covering portion 21 via the first fixing member 23.

Meanwhile, the second fluid suppression member 24 is attached to the covering portion 21 via the second fixing member 25. The first fluid suppression member 22 and the second fluid suppression member 24 have the same position in an up-down direction Y of the covering portion 21 and are disposed side by side in the left-right direction X of the covering portion 21. In addition, in the following, the left-right direction X as viewed from the subject P in a case where the subject P is wearing the examination garment 10 will be described as the left-right direction X in the covering portion 21 or in the first fluid suppression member 22, and the up-down direction Y as viewed from the subject P in a case where the subject P is wearing the examination garment 10 will be described as the up-down direction Y in the covering portion 21 or in the first fluid suppression member 22. The left-right direction X and the up-down direction Y are perpendicular to each other. An insertion direction Z, which will be described below, is a direction perpendicular to the left-right direction X and to the up-down direction Y

The second fluid suppression member 24 (a point indicated by a solid line) is disposed closer to one side of the covering portion 21 in the left-right direction X. In the present embodiment, a center line CL2 of the second fluid suppression member 24 in the left-right direction X is disposed closer to a left side with respect to the center line CL0 of the covering portion 21 in the left-right direction X (a position indicated by a solid line in FIG. 4). This is because, during an endoscopy, the subject P often takes a lateral decubitus posture in which his/her left side faces downward. Therefore, it is considered that the second fluid suppression member 24 for suction is disposed on a lower side.

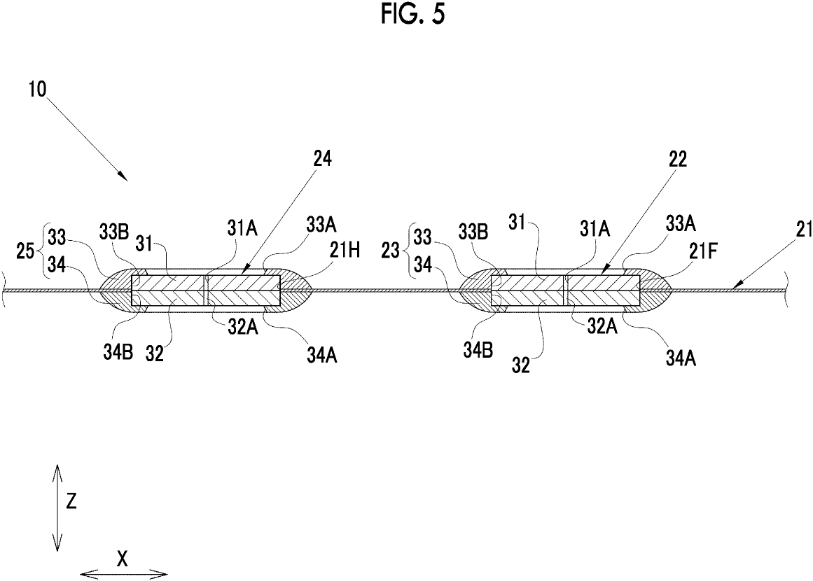

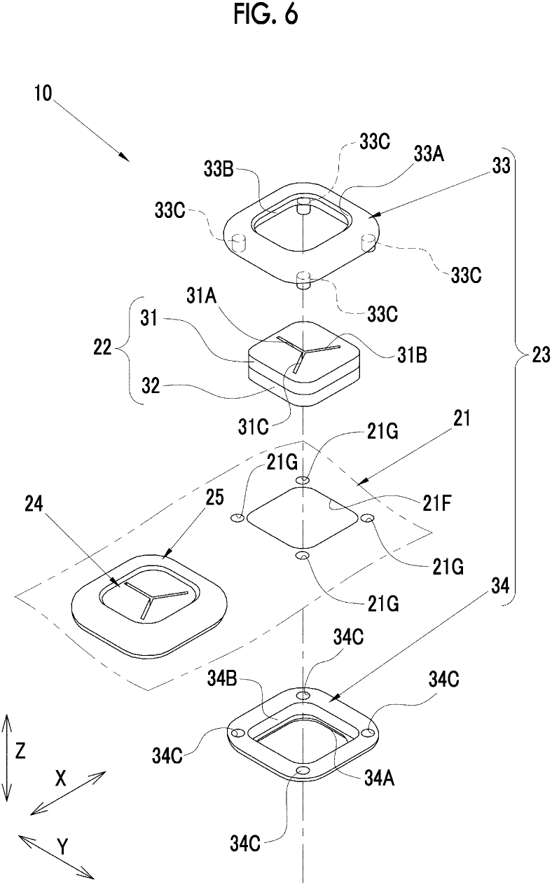

As shown in FIGS. 5 and 6, the first fluid suppression member 22 includes a first porous member 31 and a second porous member 32, and three slits are disposed in a Y shape. An insertion port 21F is formed in the covering portion 21. The insertion port 21F is a through-hole that matches the outer shape of the first fluid suppression member 22. Accordingly, since the covering portion 21 does not block the first fluid suppression member 22, the insertion part 3 can be inserted into the inside of the covering portion 21 through the insertion port 21F and the first fluid suppression member 22. A fitting hole 21G that matches a fitting pin 33C, which will be described below, is formed around the insertion port 21F.

In addition, the covering portion 21 is formed with a suction tube port 21H (refer to FIG. 5) in addition to the insertion port 21F. The suction tube port 21H is a through-hole that matches the outer shape of the second fluid suppression member 24. Although not shown, a fitting hole 21G is also formed around the suction tube port 21H, similarly to the insertion port 21F. In addition, for convenience of illustration, only the peripheries of the insertion port 21F and the suction tube port 21H (a range surrounded by a two-dot chain line) of the pants-type covering portion 21 is cut out and shown in FIG. 6.

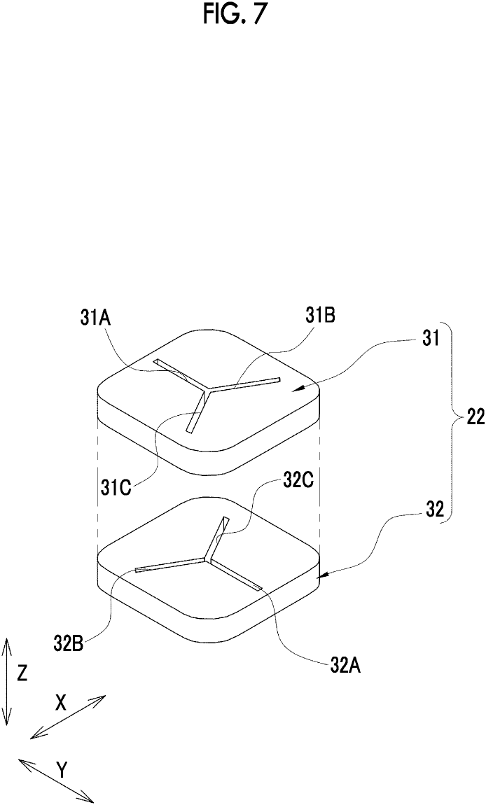

As shown in FIG. 7, the first porous member 31 has three first slits 31A to 31C. The first porous member 31 is formed by forming a flexible porous material into a quadrangular plate shape. The first slits 31A to 31C are disposed in a Y shape. More specifically, the first slits 31A to 31C are disposed at equal angle intervals of 120° and are connected to each other at the center of the first porous member 31. One first slit 31A of the first slits 31A to 31C is perpendicular to one side of the first porous member 31, that is, perpendicular to the left-right direction X, and is disposed parallel to the up-down direction Y

The second porous member 32 is formed by forming a flexible porous material into a quadrangular plate shape. The second porous member 32 has three second slits 32A to 32C. The second slits 32A to 32C are formed in a direction parallel to the insertion direction Z and intersecting the first slits 31A to 31C. Specifically, disposition of the second porous member 32 is the same as that of the first porous member 31 rotated by 180° around a central axis parallel to the insertion direction Z, and similarly to the first slits 31A to 31C, the second slits 32A to 32C are disposed at equal angle intervals of 120° and are connected to each other at the center of the second porous member 32. Accordingly, the second slits 32A to 32C are disposed at positions rotated by 180° around the central axis parallel to the insertion direction Z with respect to the first slits 31A to 31C. One second slit 32A of the second slits 32A to 32C is perpendicular to one side of the second porous member 32, that is, perpendicular to the left-right direction X, and is disposed parallel to the up-down direction Y

The porous material that forms the first and second porous members 31 and 32 is a porous material having a pore diameter and a structure that allows a gas such as air to pass therethrough and that blocks liquids, such as the body fluid, and the droplets. For example, a synthetic sponge obtained by foam-molding resin or a natural sponge such as a sea sponge is used. The first porous member 31 and the second porous member 32 may be separately formed, and may be bonded and joined in a state in which the porous members are rotated by 180° around the central axis parallel to the insertion direction Z. Cost reduction can be achieved by using two of the same porous members in this way.

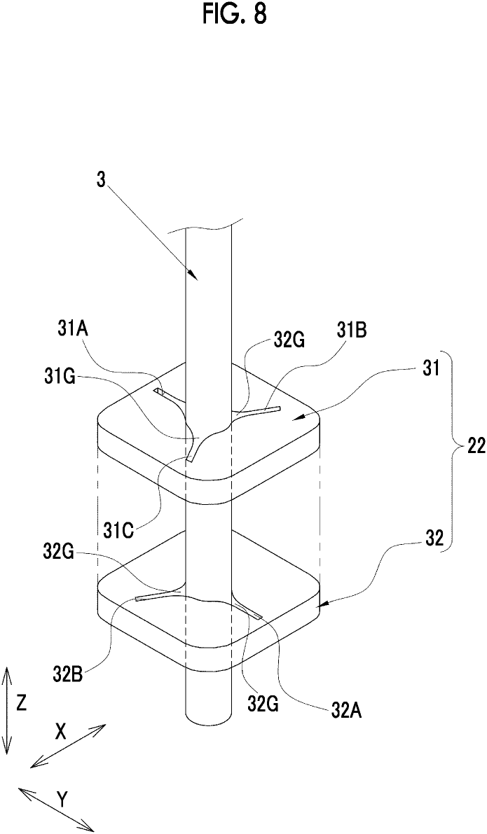

As shown in FIG. 8, in a case where the insertion part 3 is inserted into the first fluid suppression member 22, the insertion part 3 moves in the insertion direction Z while an outer peripheral surface of the insertion part 3 comes into close contact with the first slits 31A to 31C and the second slits 32A to 32C. In this case, gaps 31G and 32G are formed in some of the first slits 31A to 31C and the second slits 32A to 32C. However, as described above, since the second slits 32A to 32C are disposed by being rotated 180° around the central axis parallel to the insertion direction Z with respect to the first slits 31A to 31C, the positions of the gaps 31G and 32G do not overlap each other. Thus, it is possible to prevent the body fluid and the droplets from leaking from the gaps 31G and 32G.

In this case, by providing the first porous member 31 and the second porous member 32 with the three first slits 31A to 31C and the three second slits 32A to 32C, respectively, instead of one slit, it is possible to insert the insertion part 3 with a resistance smaller than that in a case where each porous member is provided with one slit. Moreover, it is possible to insert a smaller-diameter insertion part 3 having a diameter of about 3 mm to a large-diameter insertion part 3 having a diameter of about 16 mm with a small resistance without any gap. Accordingly, it is possible to form the first porous member 31 and the second porous member 32 from a porous material having a high density (having fewer voids), and the leakage of the body fluid and the droplets can be further suppressed.

The first fixing member 23 includes a first frame member 33 and a second frame member 34. The first frame member 33 and the second frame member 34 are formed of, for example, a soft material such as a soft resin. The first frame member 33 is disposed outside the covering portion 21, and the second frame member 34 is disposed inside the covering portion 21.

The first frame member 33 is formed in a quadrangular frame shape. The first frame member 33 has a through-hole 33A, an opening portion 33B, and four fitting pins 33C (refer to FIG. 6). The through-hole 33A exposes the first slits 31A to 31C of the first porous member 31. The opening portion 33B is an opening portion cut out in accordance with an outer peripheral surface of the first porous member 31.

The second frame member 34 is formed in a quadrangular frame shape. The second frame member 34 has a through-hole 34A, an opening portion 34B, and four fitting holes 34C (refer to FIG. 6). The through-hole 34A exposes the second slits 32A to 32C of the second porous member 32. The opening portion 34B is an opening portion cut out in accordance with an outer peripheral surface of the second porous member 32. Additionally, in each of the first frame member 33 and the second frame member 34, a surface on a side facing the covering portion 21 is formed in a planar shape, and a gently curved surface is formed to connect a surface on a side opposite to the side facing the covering portion 21 and an outer periphery to each other. Accordingly, the first frame member 33 and the second frame member 34 are not caught in the subject P or in the insertion part 3 of the endoscope 2.

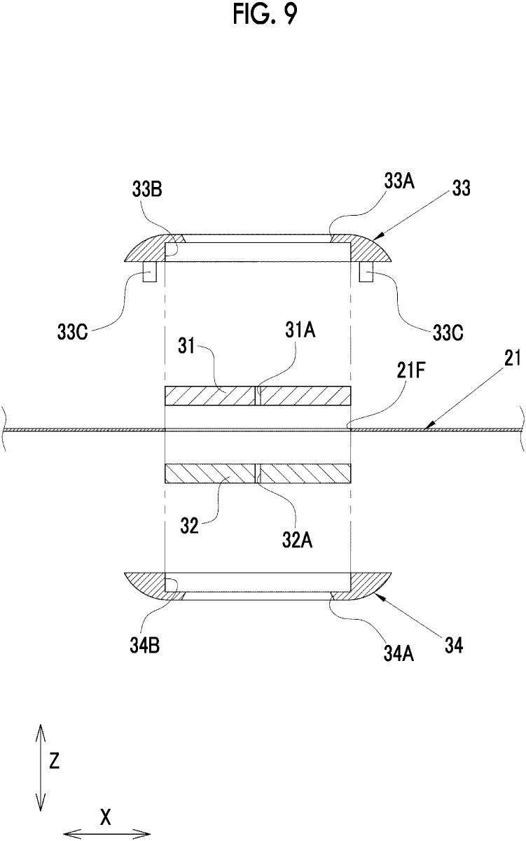

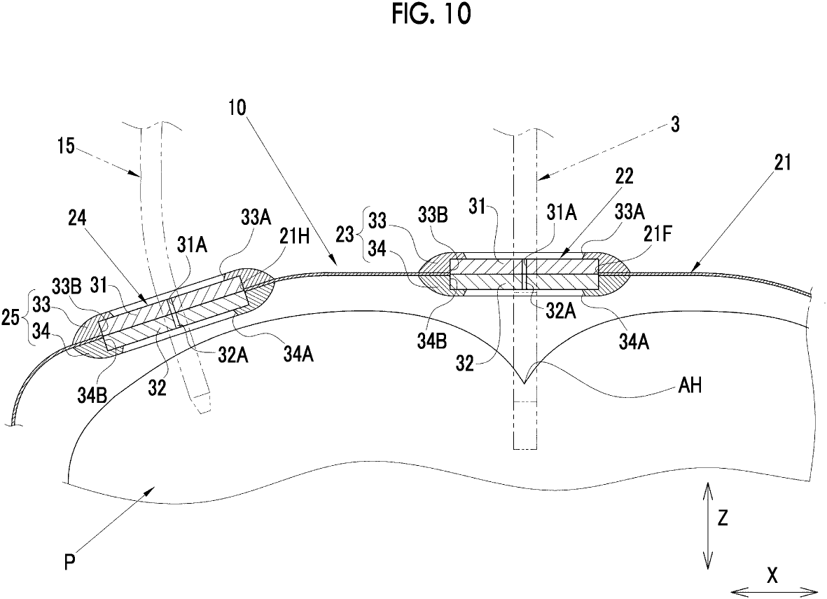

As shown in FIG. 9, in a case where the first fluid suppression member 22 is attached to the covering portion 21, the positions of the first and second porous members 31 and 32 are aligned with the position of the insertion port 21F. The opening portions 33B and 34B are fitted to the outer peripheral surfaces of the first and second porous members 31 and 32, and the covering portion 21 is sandwiched between the first frame member 33 and the second frame member 34. Accordingly, in a state in which the first porous member 31 and the second porous member 32 are laminated, the first frame member 33 and the second frame member 34 are integrally provided, and the first fluid suppression member 22 is attached to the covering portion 21. In this case, the first frame member 33 and the second frame member 34 are anchored to each other by fitting the fitting pin 33C formed in the first frame member 33 into the fitting hole 34C formed in the second frame member 34. Additionally, in a case where the first frame member 33 and the second frame member 34 sandwich the covering portion 21, the fitting pin 33C penetrates the fitting hole 21G of the covering portion 21.

In addition, the method of anchoring the first frame member 33 and the second frame member 34 to each other is not limited to the above method, and, for example, the first frame member 33 and the second frame member 34 may be anchored to each other by bonding or pressure bonding. Additionally, a fitting hole may be formed in the first frame member 33, and a fitting pin may be formed in the second frame member 34 to fit the two frame members to each other.

In addition, in the present embodiment, the first and second fluid suppression members are attached to the covering portion 21 by the first fixing member 23 in a state in which the first porous member 31 and the second porous member 32 constituting the first fluid suppression member 22 are laminated. However, the present invention is not limited to this. For example, the covering portion 21 may be directly anchored to the outer peripheral surface of the first fluid suppression member 22 that is integrally formed without providing the first fixing member 23.

The second fluid suppression member 24 has the same configuration as the first fluid suppression member 22, that is, includes the first porous member 31 and the second porous member 32, and the second slits 32A to 32C are disposed by being rotated by 180° around the central axis parallel to the insertion direction Z with respect to the first slits 31A to 31C. The second fixing member 25 has the same configuration as the first fixing member 23 and includes the first frame member 33 and the second frame member 34.

In a case where the second fluid suppression member 24 is attached to the covering portion 21, the positions of the first and second porous members 31 and 32 are aligned with the positions of the suction tube port 21H. The opening portions 33B and 34B are fitted to the outer peripheral surfaces of the first and second porous members 31 and 32, and the covering portion 21 is sandwiched between the first frame member 33 and the second frame member 34. Accordingly, the second fluid suppression member 24 is attached to the covering portion 21, similarly to the first fluid suppression member 22. In addition, the present invention is not limited to this, and the covering portion 21 may be directly anchored to the outer peripheral surface of the second fluid suppression member 24 that is integrally formed without providing the second fixing member 25. Alternatively, only one of the first fluid suppression member 22 and the second fluid suppression member 24 may be attached to the covering portion 21 by using the fixing member, and the other may be directly attached.

An operation in a case where the doctor D, who is a user, performs an endoscopy using the examination garment 10 will be described. As shown in FIG. 10, the doctor D aligns the position of the first fluid suppression member 22 with the position of an anus AH of the subject P in a state in which the subject P is wearing the examination garment 10. Additionally, in this case, the subject P is in a lateral decubitus posture in which his/her left side faces downward on the examination table T (the state shown in FIG. 1).

For the subject P who is wearing the examination garment 10, the doctor D inserts the insertion part 3 of the endoscope 2 into the body of the subject P, for example, the large intestine, from the anus AH through the first fluid suppression member 22. Additionally, the doctor D causes the suction tube 15 to enter the inside of the examination garment 10 through the second fluid suppression member 24. In addition, in this case, the doctor D may insert either the insertion part 3 or the suction tube 15 into the examination garment 10 first.

In a case where an examination is performed on the lower gastrointestinal tract such as the large intestine with the endoscope 2, the body fluids, such as excrement in the intestine and blood, and the droplets are released together with air and washing water fed through the endoscope 2. However, as described above, in the examination garment 10, the insertion part 3 is inserted into the body of the subject P through the first fluid suppression member 22. Accordingly, the first fluid suppression member 22 suppresses the passage of the fluids. Thus, it is possible to prevent the body fluid and the droplets from leaking from the insertion port 21F of the examination garment 10.

Additionally, in the case of the first fluid suppression member 22, the three first slits 31A to 31C and the three second slits 32A to 32C are guided to the centers of the first porous member 31 and the second porous member 32. Therefore, in a case where the insertion part 3 is inserted, it is not necessary to aim at the center, and the insertion can be easily performed.

Additionally, since the examination garment 10 is provided with the suction tube port 21H so that the suction tube 15 can be made to enter the inside of the examination garment 10, the body fluids inside the examination garment 10 can be suctioned through the suction tube 15 by operating the suction device 16 connected to the suction tube 15. Additionally, as shown in FIG. 11, since the suction tube port 21H and the second fluid suppression member 24 are disposed so as to be positioned on the lower side in a case where the subject P is in the lateral decubitus posture, a body fluid F stored in a lower portion of the examination garment 10 is easily suctioned.

Moreover, since the second fluid suppression member 24 is provided at the suction tube port 21H, the second fluid suppression member 24 suppresses the passage of the fluid. Thus, it is possible to prevent the body fluid and the droplets from leaking from the suction tube port 21H of the examination garment 10.

Additionally, since the covering portion 21 of the examination garment 10 is formed of a transparent or translucent sheet, the doctor D can recognize the internal state of the examination garment 10. Accordingly, the doctor D can visually recognize the amount of the body fluid stored inside the examination garment 10 through the covering portion 21. Additionally, since the examination garment 10 is in close contact with the torso portion and the thigh portion of the subject P by the string 26 passed through the string passing portion 21C, the gathering 21E, or the like, the leakage of the body fluid and the droplets from the torso hole 21A and the thigh hole 21B can be prevented.

In addition, as shown in FIG. 11, it is preferable to provide a storage portion 36 inside the covering portion 21. Accordingly, the body fluid released from the subject P can be received and stored in the storage portion 36. It is preferable that the storage portion 36 is disposed on the left side of the covering portion 21 in the left-right direction X. Accordingly, in a case where the subject P who is wearing the examination garment 10 is in a lateral decubitus posture in which his/her left side faces downward, it is easy to store the body fluid because the storage portion 36 is located at the lower portion of the examination garment 10. It is preferable that the storage portion 36 has, for example, a bowl-shaped container shape.

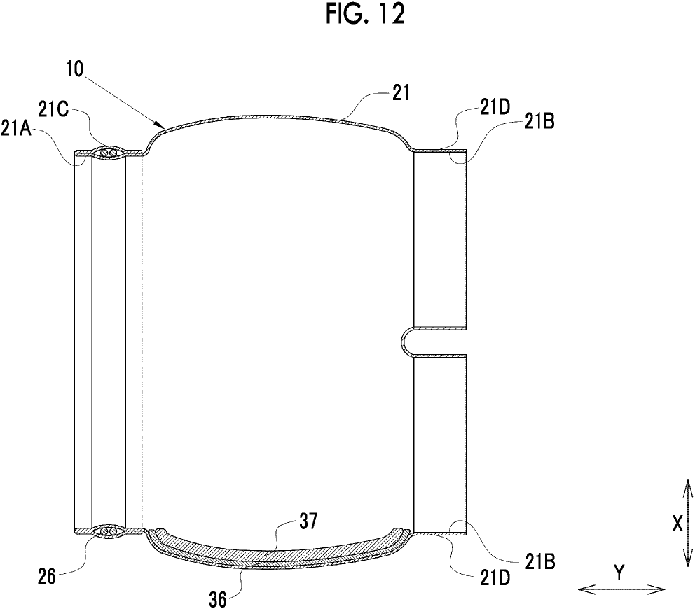

Additionally, as shown in FIG. 12, the storage portion 36 and an absorbent material 37 may be provided in a double structure inside the covering portion 21. The absorbent material 37 is disposed inside the storage portion 36. As the absorbent material 37, it is preferable to use a so-called polymer absorbent material used for diapers or the like. By adopting such a structure, the body fluid can be received, similarly to the example shown in FIG. 11, and the absorbent material 37 can absorb and coagulate the body fluid. Thus, it is possible to further prevent the body fluid from leaking to the outside of the examination garment 10. In addition, the present invention is not limited to this, and the absorbent material 37 may be directly anchored to the covering portion 21.

Additionally, a discolored portion of which the color changes by absorbing the liquid may be provided on at least a part of the absorbent material 37. The discolored portion is formed of, for example, a color-developing ink that is transparent during drying and that develops a color in a case where the discolored portion is wetted with a liquid, which is used for diapers or the like. Accordingly, the doctor D can easily visually recognize a state in which the absorbent material 37 has absorbed the body fluid. Additionally, as the discolored portion, the absorbent material 37 may be provided with a water-soluble resin layer that is opaque during drying, which becomes transparent or translucent in a case where the discolored portion is wetted with water, and one in which a colored ink layer is laminated on the water-soluble resin layer. Accordingly, in a case where the water-soluble resin layer is wetted with the liquid, the water-soluble resin layer becomes transparent, and the colored ink layer on the resin layer can be visually recognized. Additionally, the discolored portion is not limited to these examples, and it is preferable to use a material of which the color changes by absorbing a liquid, such as using a dye of which the color changes depending on the pH (hydrogen ion concentration index) of an aqueous solution.

Additionally, the discolored portion formed of a material that is discolored by absorbing a liquid as described above may be provided separately from the absorbent material 37, and the absorbent material 37 and the above discolored portion may be laminated and anchored to the covering portion 21. In addition, it is preferable that the absorbent material 37 is attachable to and detachable from the covering portion 21, for example, is fixed with a double-sided tape having a pressure-sensitive adhesive force such that the tape can be peeled off. Accordingly, the absorbent material 37 that has absorbed the body fluid and the covering portion 21 can be separated and discarded.

### Second Embodiment

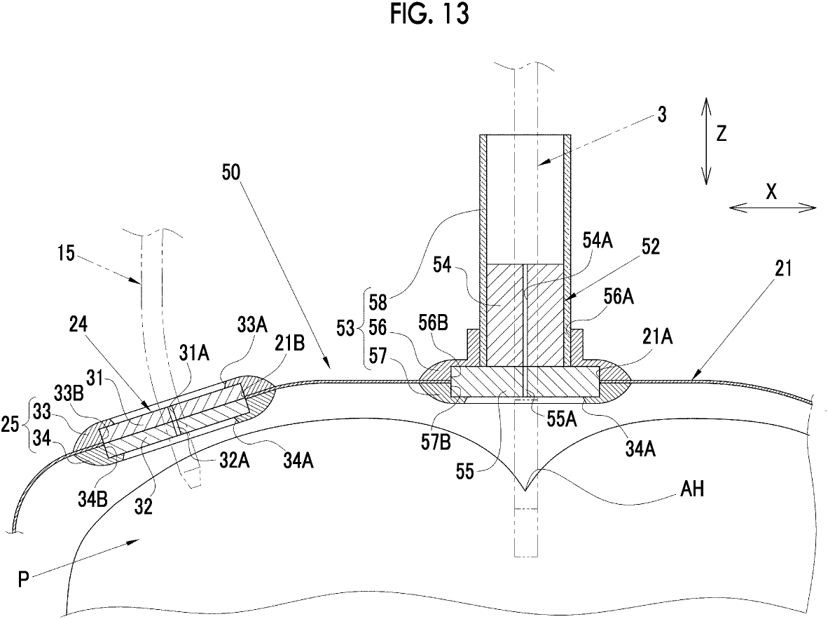

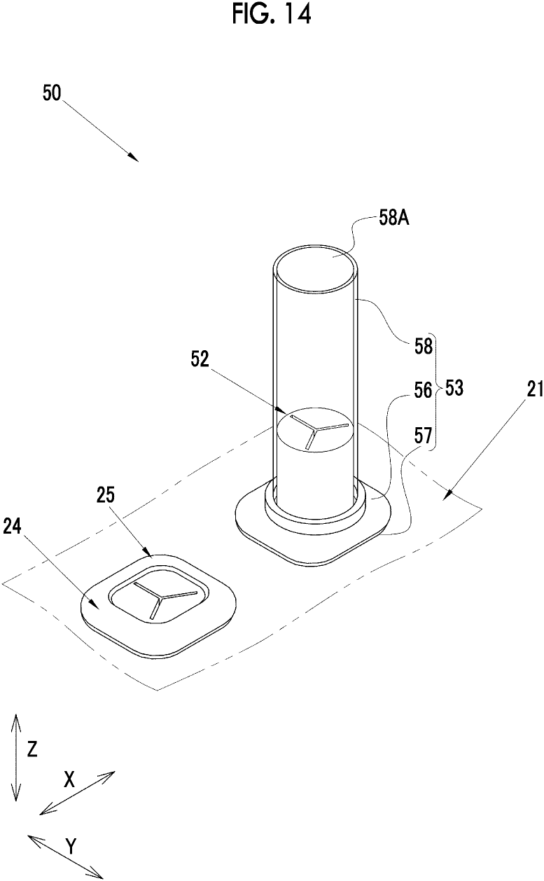

In the above first embodiment, the first fluid suppression member 22 and the second fluid suppression member 24 are made of the same components, and the first fixing member 23 and the second fixing member are made of the same components. However, the present invention is not limited to this, and different components may be used for both of the fluid suppression members. As shown in FIGS. 13 and 14, an examination garment 50 comprises the covering portion 21, a first fluid suppression member 52, a first fixing member 53, the second fluid suppression member 24, and the second fixing member 25. The first fluid suppression member 52 and the first fixing member 53 are made of components that are different from the first fluid suppression member 22 and the first fixing member 23 of the above first embodiment. In addition, the present embodiment has the same configuration as that of the examination garment 10 of the above first embodiment except that the fluid suppression member 52 is used instead of the fluid suppression member 22, and the first fixing member 53 is used instead of the first fixing member 23, and the same components will be designated by the same reference numerals, and the description thereof will be omitted.

The first fluid suppression member 52 is disposed at the center of the covering portion 21 on the rear surface side, similarly to the first fluid suppression member 22 of the above first embodiment. The first fluid suppression member 52 is attached to the covering portion 21 via the first fixing member 53.

Meanwhile, the second fluid suppression member 24 is attached to the covering portion 21 via the second fixing member 25, similarly to the above first embodiment. The first fluid suppression member 52 and the second fluid suppression member 24 have the same position in the up-down direction Y of the covering portion 21 and are disposed side by side in the left-right direction X of the covering portion 21. The second fluid suppression member 24 is disposed closer to the left side of the covering portion 21 in the left-right direction X, similarly to the above first embodiment.

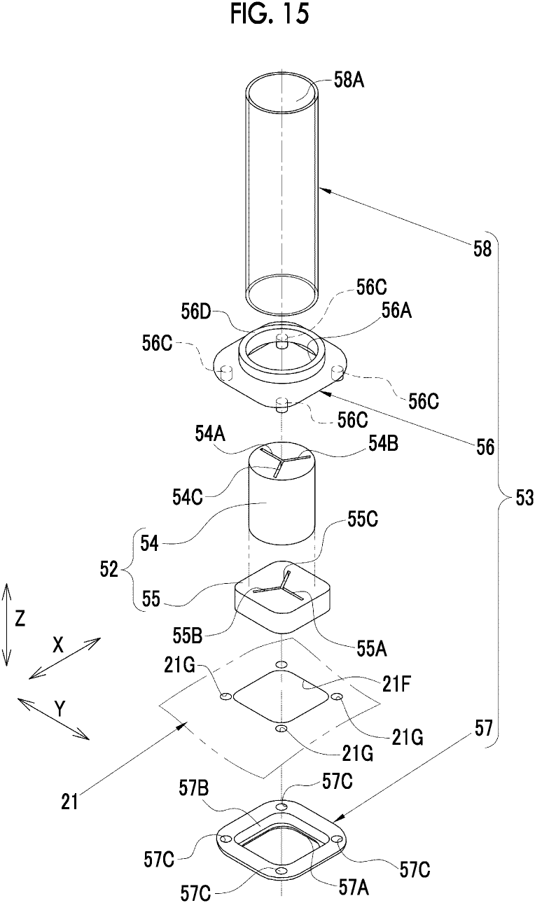

As shown in FIG. 15, the first fluid suppression member 52 has a first porous member 54 and a second porous member 55. The first porous member 54 is formed by forming a flexible porous material into a columnar shape. The first porous member 54 has a longer axial dimension than a radial dimension. The first porous member 54 has three first slits 54A to 54C. The first slits 54A to 54C are disposed in a Y shape. Specifically, the first slits 54A to 54C are disposed at equal angle intervals of 120° and are connected to each other at the center of the first porous member 54.

The second porous member 55 is formed by forming a flexible porous material into a quadrangular plate shape. The second porous member 55 has three second slits 55A to 55C. The second slits 55A to 55C are formed in a direction parallel to the insertion direction Z and intersecting the first slits 54A to 54C. Specifically, similarly to the first slits 54A to 54C, the second slits 55A to 55C are disposed at equal angle intervals of 120° and are connected at the center of the second porous member 55. Accordingly, the second slits 55A to 55C are disposed at positions rotated by 180° around the central axis parallel to the insertion direction Z with respect to the first slits 54A to 54C.

The porous materials that form the first porous member 54 and the second porous member 55 are the same as the porous materials that form the first and second porous members 31 and 32 of the above first embodiment. It is preferable that the first porous member 54 and the second porous member 55 are separately formed and joined by bonding or the like in a state in which the second slits 55A to 55C are rotated by 180° around the central axis parallel to the insertion direction Z with respect to the first slits 54A to 54C.

In a case where the first porous member 54 is attached to the covering portion 21, the first porous member 54 has smaller dimensions in the left-right direction X and in the up-down direction Y than the second porous member 32 and protrudes in the insertion direction Z with respect to the second porous member 32 (refer to FIG. 15). Thus, in a case where the subject P is wearing the covering portion 21, that is, in a case where the covering portion 21 has covered the subject P, the first porous member 54 forms a convex portion that protrudes to the outside of the covering portion 21. By adopting the shape having the convex portion, the first fluid suppression member 52 can be formed to have a longer dimension in the insertion direction Z than the first fluid suppression member 22 of the above first embodiment. Thus, the passage of the fluid can be sufficiently suppressed, and the insertion part 3 can be smoothly inserted.

The first fixing member 53 includes a first frame member 56, a second frame member 57, and a tubular member 58. The first frame member 56, the second frame member 57, and the tubular member 58 are formed of, for example, a soft material such as a soft resin. The first frame member 56 and the tubular member 58 are disposed outside the covering portion 21, and the second frame member 57 is disposed inside the covering portion 21.

The first frame member 56 is formed in a quadrangular frame shape. The first frame member 56 has a through-hole 56A, an opening portion 56B, and four fitting pins 56C (refer to FIG. 15). The through-hole 56A is a circular through-hole whose inner diameter matches the outer diameter of the tubular member 58. The opening portion 56B is an opening portion cut out in accordance with an outer peripheral surface of the second porous member 55. Additionally, a peripheral edge of the through-hole 56A is formed with a convex portion 56D that is one step more convex than the periphery in the insertion direction Z.

The tubular member 58 is formed in a cylindrical shape having a pipe line 58A into which the insertion part 3 is inserted. The pipe line 58A is a through-hole whose inner diameter matches the outer diameter of the first porous member 54. Additionally, it is preferable that the tubular member 58 is made of a transparent resin. This is because the insertion state of the insertion part 3 can be visually recognized from the outside. The tubular member 58 is anchored to the first frame member 56 by fitting an outer peripheral surface of a lower end part thereof to the through-hole 56A of the first frame member 56. Accordingly, the tubular member 58 extends to the outside of the covering portion 21 in a case where the subject P is wearing the covering portion 21, that is, in a case where the covering portion 21 has covered the subject P.

The second frame member 57 is formed in a quadrangular frame shape. The second frame member 57 has a through-hole 57A, an opening portion 57B, and four fitting holes 57C (refer to FIG. 6). The through-hole 57A exposes the second slits 55A to 55C of the second porous member 55. The opening portion 57B is an opening portion cut out in accordance with an outer peripheral surface of the second porous member 55. Additionally, in each of the first frame member 56 and the second frame member 57, a surface on a side facing the covering portion 21 is formed in a planar shape, and a gently curved surface is formed to connect a surface on a side opposite to the side facing the covering portion 21 and an outer periphery to each other. Accordingly, the first frame member 56 and the second frame member 57 are not caught in the subject P or in the insertion part 3 of the endoscope 2.

In a case where the first fluid suppression member 52 is attached to the covering portion 21, the positions of the first porous member 54 and the second porous member 55 are aligned with the position of the insertion port 21F. The pipe line 58A of the tubular member 58 is fitted to an outer peripheral surface of the first porous member 54, the opening portions 56B and 57B are fitted to an outer peripheral surface of the second porous member 55, and the covering portion 21 is sandwiched between the first frame member 56 and the second frame member 57. Accordingly, in a state in which the first porous member 54 and the second porous member 55 are laminated, the first frame member 56 and the second frame member 57 are integrally provided, and the first fluid suppression member 52 is attached to the covering portion 21.

In this case, the first frame member 56 and the second frame member 57 are anchored to each other by fitting the fitting pin 56C formed in the first frame member 56 into the fitting hole 57C formed in the second frame member 57. Additionally, in a case where the first frame member 56 and the second frame member 57 sandwich the covering portion 21, the fitting pin 56C penetrates the fitting hole 21G of the covering portion 21.

In addition, the method of fixing the first frame member 56 and the second frame member 57 to each other is not limited to the above method, and for example, the first frame member 56 and the second frame member 57 may be anchored to each other by bonding or pressure bonding. Additionally, a fitting hole may be formed in the first frame member 56, and a fitting pin may be formed in the second frame member 57 to fit the two frame members to each other.

An operation in a case where the doctor D, who is a user, performs an endoscopy using the examination garment 50 will be described. For the subject P who is wearing the examination garment 50, the doctor D inserts the insertion part 3 of the endoscope 2 into the body of the subject P, for example, the large intestine, from the anus AH through the first fluid suppression member 22. In this case, since the insertion part 3 is guided by the tubular member 58 and the inside of the tubular member 58 can be visually recognized, the insertion can be easily performed. Additionally, the doctor D causes the suction tube 15 to enter the inside of the examination garment 50 through the second fluid suppression member 24.

In the examination garment 50, the first fluid suppression member 52 suppresses the passage of the fluid. Since the first fluid suppression member 52 has a longer dimension in the insertion direction Z than the fluid suppression member 22 of the above first embodiment, it is possible to more reliably prevent the body fluid and the droplets from leaking from the insertion port 21F of the examination garment 50. Additionally, the examination garment 50 is provided with the suction tube port 21H so that the body fluid or the like inside the examination garment 10 can be suctioned through the suction tube 15. Additionally, since the second fluid suppression member 24 is provided, it is possible to prevent the body fluid and the droplets from leaking from the suction tube port 21H.

Additionally, although not shown, similarly to the examination garment 10 of the above first embodiment, it is preferable that the storage portion 36 is provided inside the covering portion 21 in the examination garment 50 of the present embodiment as well. Additionally, the storage portion 36 and the absorbent material 37 may be provided in a double structure inside the covering portion 21. In addition, the absorbent material 37 may be directly anchored to the covering portion 21. It is preferable that the absorbent material 37 is attachable to and detachable from the covering portion 21. Additionally, it is preferable that a discolored portion of which the color changes by absorbing the liquid is used for at least a part of the absorbent material 37. Additionally, the discolored portion may be provided separately from the absorbent material 37, and an absorbent and the discolored portion may be laminated and anchored to the covering portion 21.

### First Modification Example

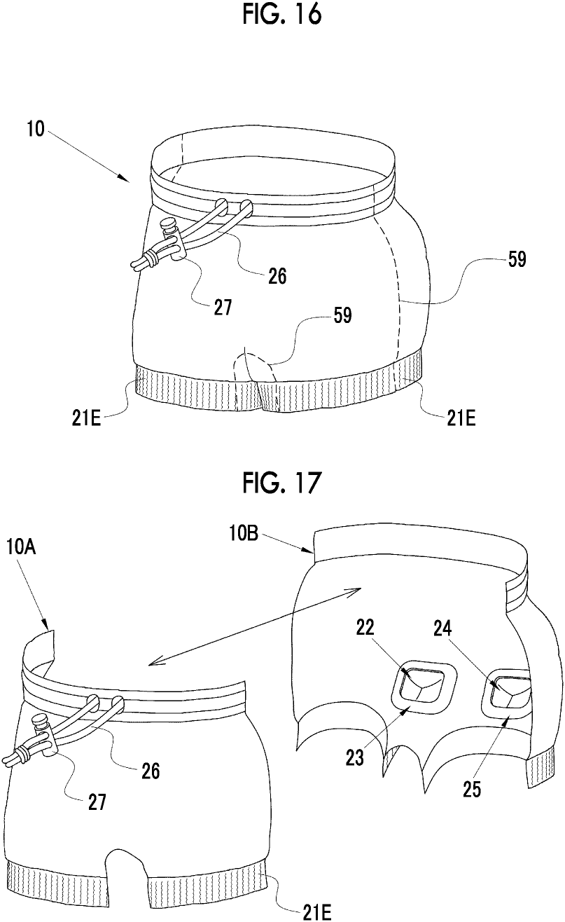

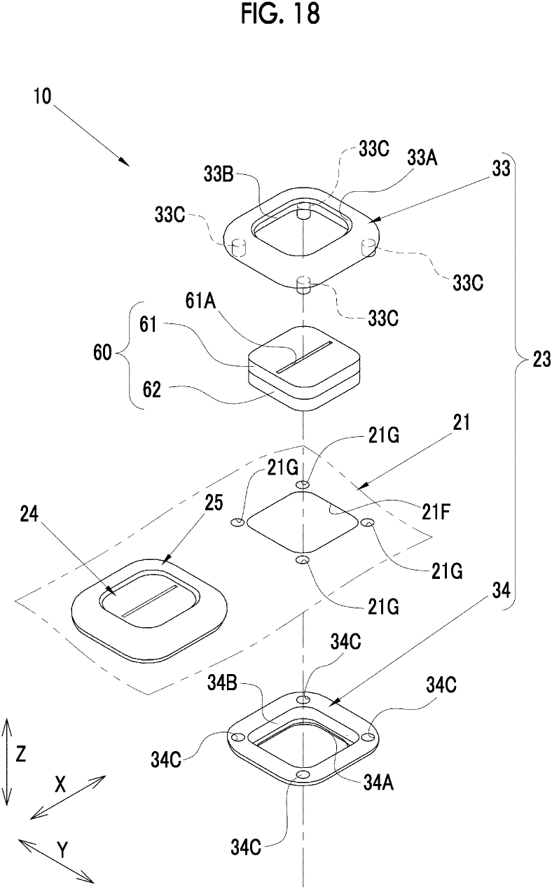

Additionally, in the above first and second embodiments, it is not considered that the subject P takes off the examination garments 10 and 50 after the endoscopy. However, in a modification example shown in FIG. 16, a fragile portion 59 is provided on a side surface portion and on a thigh portion of the covering portion 21. The fragile portion 59 is a portion in which a part of the covering portion 21 is fragilely formed like perforations so that the fragile portion is separable by a user’s force (general human force). In a case where the subject P, the doctor D, or the helper cuts the covering portion 21 along the fragile portion 59, as shown in FIG. 17, the examination garments 10 and 50 can be divided into a front surface portion 10A and a rear surface portion 10B. Therefore, the subject P can easily take off the examination garments 10 and 50. In addition, as such a fragile portion 59, it is sufficient to provide a portion processed so that the covering portion 21 can be easily separated by a user’s force. For example, a portion where the thickness of the covering portion 21 is just partially reduced or a portion bonded with an adhesive with a weak pressure-sensitive adhesive force may be provided.

### Second Modification Example

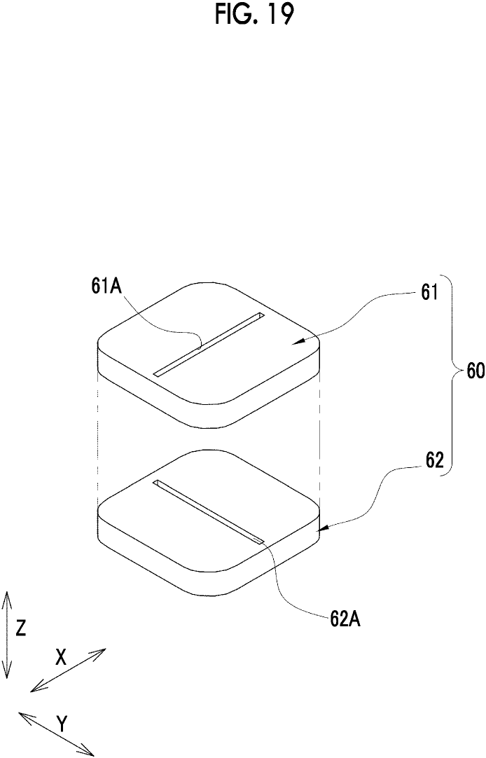

Additionally, in the above first and second embodiments, an example is given in which the first and second fluid suppression members include first and second porous members in which three slits disposed in a Y shape are formed. However, the present invention is not limited to this, and in a modification example shown in FIG. 18, a fluid suppression member 60 includes first and second porous members 61 and 62 in which slits are formed one by one.

The first porous member 61 is formed by forming a flexible porous material into a quadrangular plate shape. The first porous member 61 has a first slit 61A. The first porous member 61 fits into the opening portion 33B of the first frame member 33. The first slit 61A is formed parallel to the left-right direction X and parallel to the insertion direction Z of the insertion part 3.

As shown in FIG. 19, the second porous member 62 is formed by forming a flexible porous material into a quadrangular plate shape. The second porous member 62 has a second slit 62A. The second porous member 62 fits into the opening portion 34B of the second frame member 34. The second slit 62A is formed in a direction parallel to the insertion direction Z and intersecting the first slit 61A. More specifically, the second slit 62A is formed parallel to the insertion direction Z and parallel to the up-down direction Y In this way, since the first slit 61A and the second slit 62A intersect each other, the positions of the gaps with respect to the insertion part 3 do not overlap each other. Thus, the fluid suppression member 60 can suppress the passage of the fluid and can prevent the body fluid and the droplets from leaking from the fluid suppression member 60.

In addition, in the present modification example, the disposition of the second porous member 62 may be the same as that of the first porous member 61 rotated by 90° around the central axis parallel to the insertion direction Z. In addition, in this case, it is preferable that the outer shapes of the first and second porous members 61 and 62 are both square.

The porous materials that form the first and second porous members 61 and 62 are the same as the porous materials that form the first and second porous members 31 and 32 of the above first embodiment. It is preferable that the first porous member 61 and the second porous member 62 are separately formed and joined by bonding or the like in a state in which the second slit 62A is disposed in a direction intersecting the first slit 61A.

In addition, the fluid suppression member 60 may be used for any of the first and second fluid suppression members in the above first and second embodiments. Additionally, similarly to the above first and second embodiments, it is preferable that the first and second fixing members are attached to the covering portion 21. Additionally, as in the above second embodiment, one of the first and second porous members 61 and 62 may be formed to have a longer dimension in the insertion direction Z, or one of the first and second porous members 61 and 62 may be formed in a columnar shape. Moreover, the dimensions of the first and second fixing members in the insertion direction Z may be increased in accordance with the dimensions of the first and second porous members 61 and 62.

### Third Modification Example

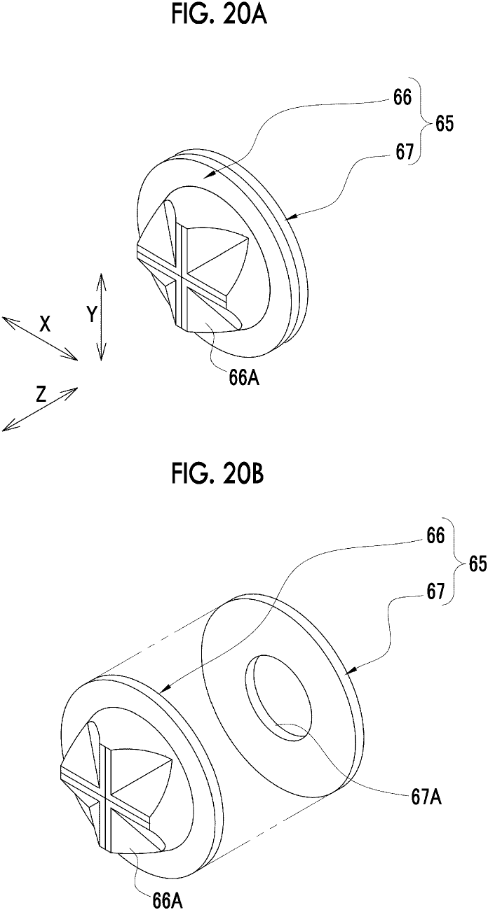

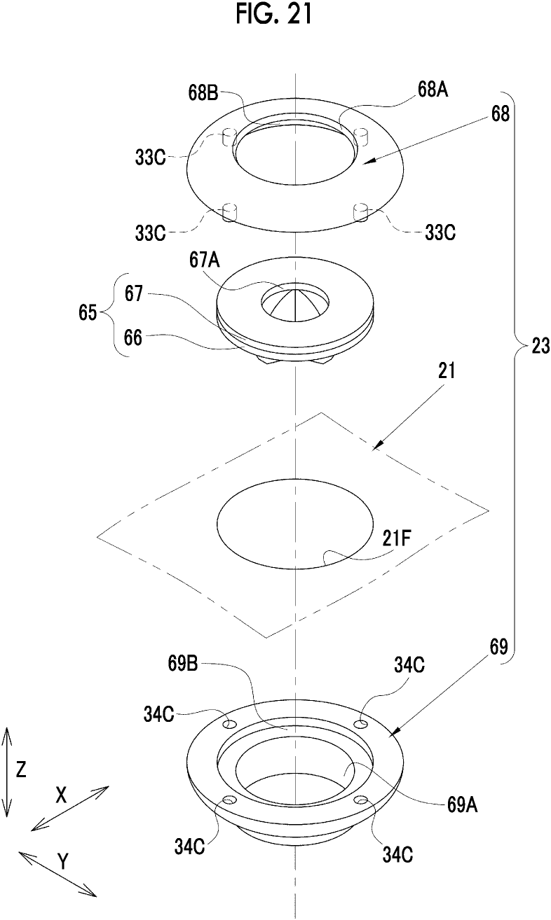

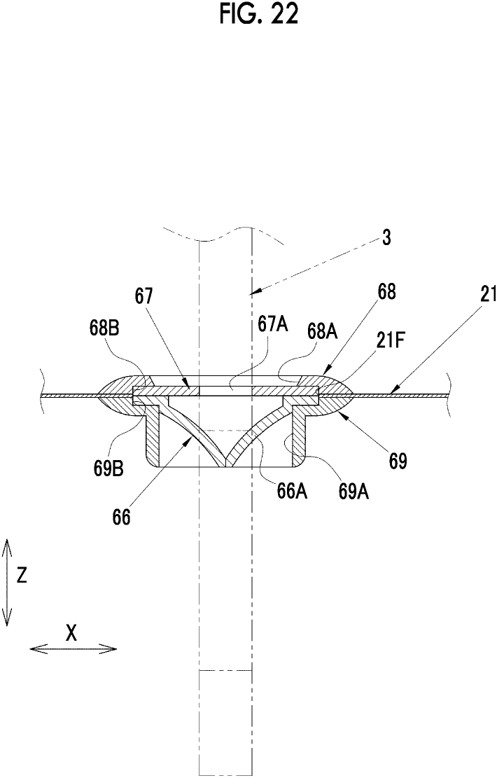

In the above first and second embodiments, an example in which the first and second fluid suppression members 22 and 24 include the first and second porous members 31 and 32 in which slits are formed and the like has been shown. However, the present invention is not limited to this as long as the fluid suppression members can suppress the passage of the fluid in both a state in which the insertion part 3 is inserted and a state in which the insertion part 3 is not inserted. FIGS. 20A and 20B show the configuration of a fluid suppression member 65 having a duckbill valve 66 and a thin plate member 67. As shown in FIG. 21, the fluid suppression member 65 is attached to the insertion port 21F of the covering portion 21 in a state in which the duckbill valve 66 and the thin plate member 67 are laminated.

As shown in FIG. 20A, the duckbill valve 66 has a well-known configuration used for an endoscope insertion guide or the like and has a plurality of lip portions 66A that protrudes in the insertion direction Z. The duckbill valve 66 is formed of an elastic member such as rubber. Accordingly, in the duckbill valve 66, the lip portions 66A are brought into close contact with each other due to an elastic force and are closed, in a state in which the insertion part 3 is not inserted. That is, the passage of the fluid in the insertion port 21F is suppressed. In addition, in a case where the insertion part 3 is inserted through the duckbill valve 66, the lip portions 66A are brought into an open state against the elastic force, and the insertion part 3 is allowed to be inserted.

Meanwhile, as shown in FIG. 20B, the thin plate member 67 has a through-hole 67A having an inner diameter that matches the outer diameter of the insertion part 3. The thin plate member 67 is formed of an elastic member such as rubber. Accordingly, in the thin plate member 67, an inner peripheral surface of the through-hole 67A is in close contact with the outer peripheral surface of the insertion part 3 in a state in which the insertion part 3 is inserted. That is, the passage of the fluid in the insertion port 21F is suppressed.

In this modification example, since the duckbill valve 66 and the thin plate member 67 constituting the fluid suppression member 65 have a circular outer shape, it is preferable to use circular first and second frame members 68 and 69 as the first fixing member 23 to be attached to the insertion port 21F of the covering portion 21 (refer to FIG. 21). In addition, the first and second frame members 68 and 69 are the same as the first and second frame members 33 and 34 of the above first embodiment except that the through-holes 68A and 69A and the opening portions 68B and 69B have a circular shape, and the description thereof will be omitted.

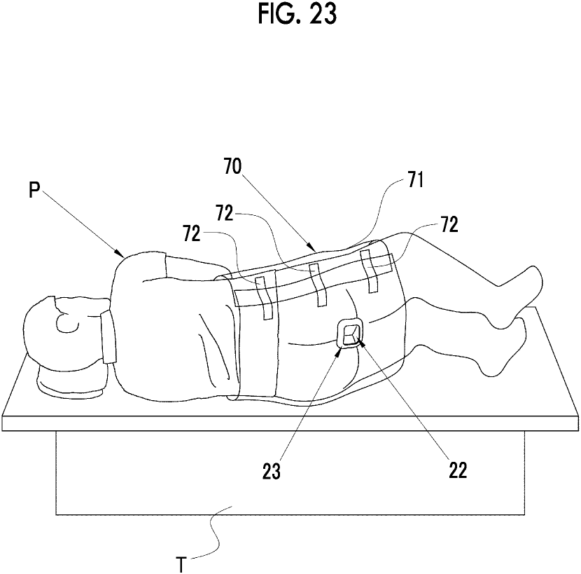

As shown in FIG. 22, similarly to the above first embodiment, the opening portions 68B and 69B are fitted to outer peripheral surfaces of the duckbill valve 66 and the thin plate member 67, and the covering portion 21 is sandwiched between the first and second frame members 68 and 69. Accordingly, in a state in which the duckbill valve 66 and the thin plate member 67 are laminated, the first frame member 68 and the second frame member 69 are integrally provided, and the fluid suppression member 65 is attached to the covering portion 21.

By virtue of the above configuration, even in a case where the insertion part 3 is inserted through the fluid suppression member 65 or even in a case where the insertion part 3 is not inserted, the passage of the fluid can be suppressed, and the body fluid and the droplets can be prevented from leaking from the inside of the examination garment to the outside thereof through the fluid suppression member 65. In addition, the fluid suppression member 65 may be used for any of the first and second fluid suppression members in the above first and second embodiments. Additionally, as in the above second embodiment, one of the first and second fixing members may be formed to have a longer dimension in the insertion direction Z.

### Third Embodiment

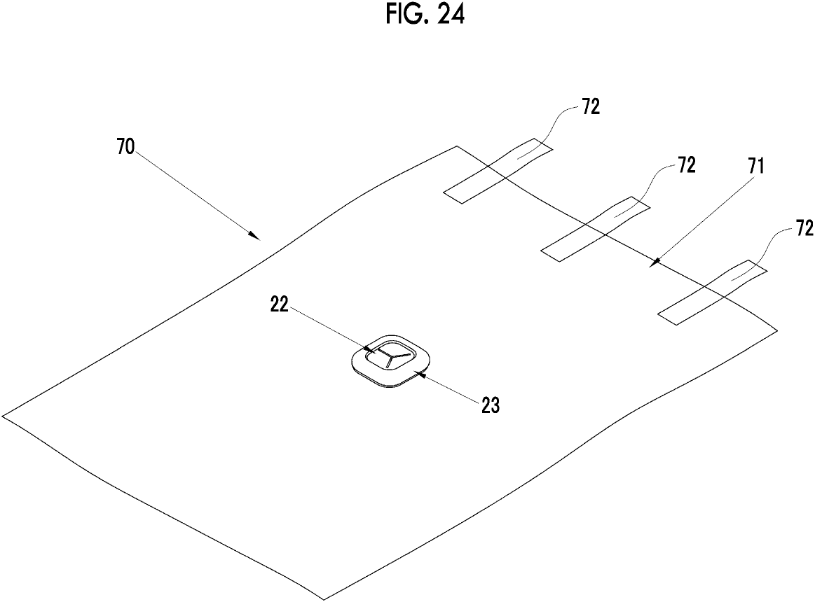

In the above first and second embodiments and various modification examples, examples in which the pants-type covering portion is used are given, but the present invention is not limited thereto. As shown in FIG. 23, an examination garment 70 includes a covering portion 71, the first fluid suppression member 22, the first fixing member 23, and a pressure-sensitive adhesive tape 72. In addition, FIG. 23 shows a state before the subject P undergoes an endoscopy. The subject P wears the examination garment 70 and is in a lateral decubitus posture in which his/her left side faces downward on the examination table T.

The covering portion 71 is formed of a planar shape sheet that can be wrapped around the subject P, who is a test object. Similarly to the covering portion 21 of the above first and second embodiments, the covering portion 71 is a transparent or translucent member and is formed of, for example, a transparent or translucent vinyl sheet having flexibility. In the present embodiment, the covering portion 71 has a size to cover at least a part of the subject P from the torso portion to the thigh portion. In addition, on the upper body of the subject P that is not covered with the covering portion 71, an examination garment for the upper body may be worn separately from the examination garment 70.

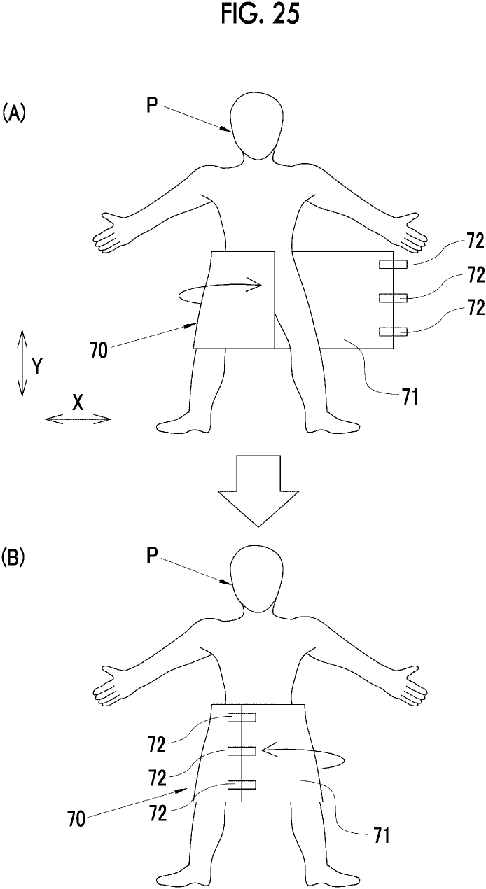

As shown in FIG. 24, the covering portion 71 can be brought into an unfolded state in a case where the covering portion 71 is not wrapped around the subject P, and is a sheet having a quadrangular outer shape in a case where the covering portion 71 is brought into the unfolded state. The first fluid suppression member 22 is located at the center of the covering portion 71. The pressure-sensitive adhesive tape 72 serving as a holding member is caused to adhere to one side of the covering portion 71. The pressure-sensitive adhesive tape 72 is caused to adhere such that at least a part of the pressure-sensitive adhesive tape 72 protrudes from one side of the covering portion 71 in a case where the covering portion 71 is brought into the unfolded state.

The covering portion 71 is formed with the insertion port 21F, similarly to the covering portion 21 in the above first and second embodiments (not shown). In a case where the first fluid suppression member 22 is attached to the covering portion 71, the positions of the first and second porous members 31 and 32 are aligned with the position of the insertion port 21F. The opening portions 33B and 34B are fitted to the outer peripheral surfaces of the first and second porous members 31 and 32, and the covering portion 71 is sandwiched between the first frame member 33 and the second frame member 34. Accordingly, the first fluid suppression member 22 is attached to the covering portion 71, similarly to the above first and second embodiments.

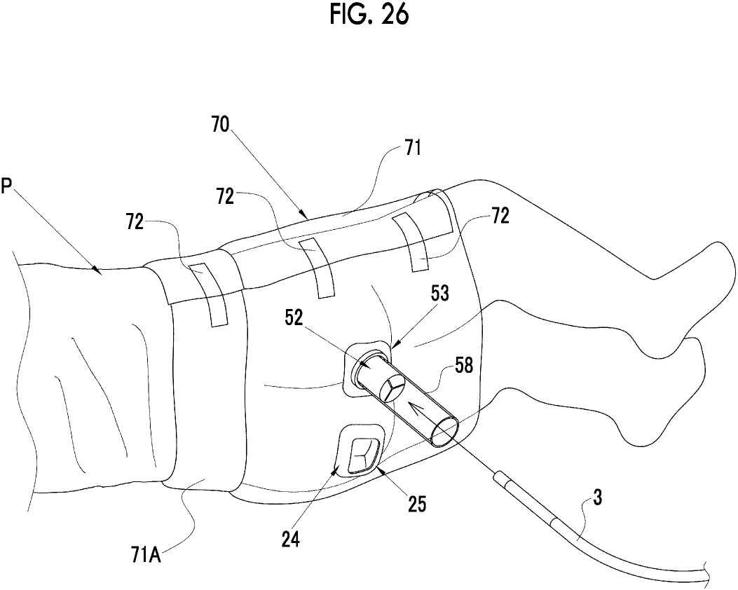

With reference to FIG. 25, a method of having the subject P wear the examination garment 70, specifically, a method of wrapping the covering portion 71 around the subject P and a method of causing the pressure-sensitive adhesive tape 72 to adhere, will be described. As shown in (A) of FIG. 25, the doctor D or the helper wraps the covering portion 71 around the torso portion and the thigh portion from one side of the covering portion 71 with respect to the subject P and from the one side on which the pressure-sensitive adhesive tape 72 is not caused to adhere. In this case, it is preferable that the covering portion 71 is disposed parallel to the left-right direction X of the subject P. Additionally, although not shown, it is preferable to align the position of the first fluid suppression member 22 with the position of the anus AH.

Next, as shown in (B) of FIG. 25, the covering portion 71 is wrapped around the torso portion and the thigh portion of the subject P from one side on which the pressure-sensitive adhesive tape 72 is caused to adhere to the subject P. A portion of the pressure-sensitive adhesive tape 72 that protrudes from one side of the covering portion 71 adheres to the covering portion 71. Accordingly, the pressure-sensitive adhesive tape 72 can hold a state in which the covering portion 71 is wrapped around the subject P.

For the subject P that is wearing the examination garment 70, similarly to the above first and second embodiments, the doctor D inserts the insertion part 3 of the endoscope 2 into the body of the subject P, for example, the large intestine, from the anus AH through the first fluid suppression member 22 located at the insertion port 21F. Similarly to the above first and second embodiments, the first fluid suppression member 22 suppresses the passage of the fluid. Thus, it is possible to prevent the body fluid and the droplets from leaking from the insertion port 21F of the examination garment 70.

### Fourth Modification Example

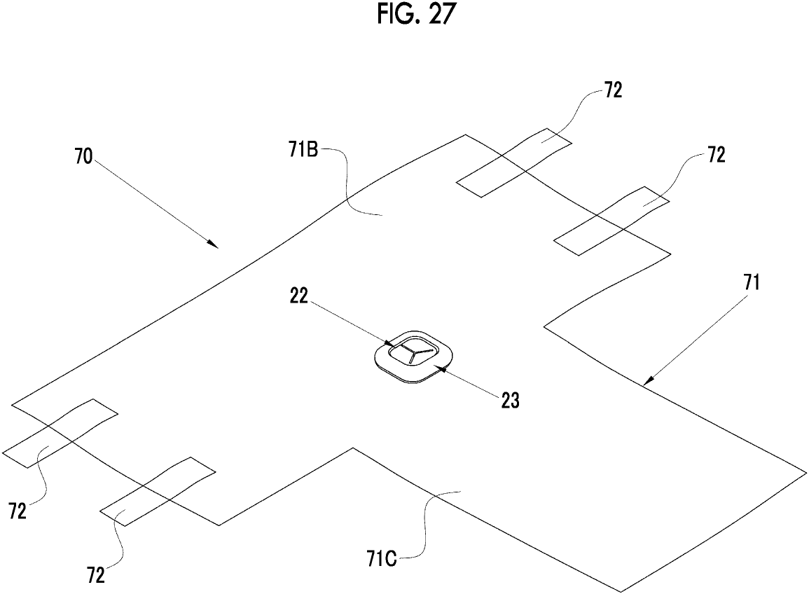

In the above third embodiment, the covering portion 71 is provided with only the first fluid suppression member 22 for insertion. However, the present invention is not limited to this, and as shown in FIG. 26, the second fluid suppression member 24 for suction may be provided. In this case, the suction tube port 21H is formed in the covering portion 71, and similarly to the above first and second embodiments, it is preferable that the covering portion 71 is sandwiched by the second fixing member 25, and the second fluid suppression member 24 is attached to the position of the suction tube port 21H. Additionally, similarly to the above first and second embodiments, it is preferable that the second fluid suppression member 24 is disposed closer to the left side of the covering portion 71 in the left-right direction X.

Additionally, the first suppression member and the first fixing member attached to the covering portion 71 may be the first fluid suppression member 52 and the first fixing member 53 in the second embodiment. Accordingly, similarly to the above second embodiment, since the insertion part 3 is guided by the tubular member 58, and the inside of the tubular member 58 can be visually recognized, the insertion can be easily performed. Additionally, since the first fluid suppression member 52 has a long dimension in the insertion direction Z, the passage of the fluid can be sufficiently suppressed, and the insertion part 3 can be smoothly inserted.

Additionally, as shown in FIG. 26, it is preferable that the gathering 71A is located at a point located on the torso portion of the subject P in a case where the covering portion 71 is wrapped around the subject P, that is, in a case where the covering portion 71 covers the subject P. The gathering 71A has contractility, similarly to the gathering 21E of the covering portion 21 in the above first embodiment. Accordingly, since the covering portion 71 is in close contact with the torso portion of the subject P, leakage of the body fluid and the droplets from the periphery of the torso portion of the subject P can be prevented.

### Fifth Modification Example

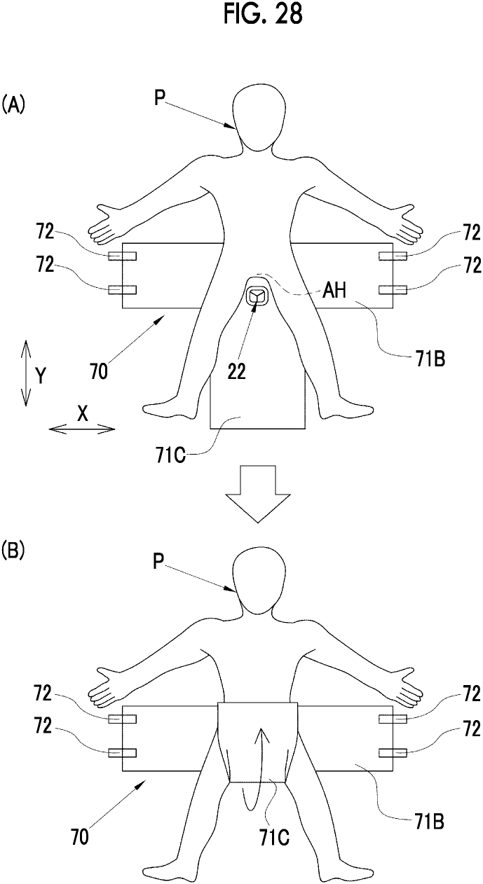

Additionally, in the above third embodiment, the covering portion 71 is a sheet having a quadrangular outer shape in a case where the covering portion 71 is brought into the unfolded state. However, the present invention is not limited to this, and as shown in FIG. 27, the covering portion 71 may be a sheet having a T-shaped outer shape in a case where the covering portion 71 is brought into the unfolded state. In this case, it is preferable that the first fluid suppression member 22 is located near the center of the covering portion 71, specifically, at a portion where a horizontal line portion 71B and a vertical line portion 71C of the covering portion 71 that form a T-shape intersect each other.

The pressure-sensitive adhesive tape 72 is caused to adhere to both end parts of the horizontal line portion 71B of the covering portion 71 that forms the T-shape. The pressure-sensitive adhesive tape 72 is caused to adhere such that at least a part of the pressure-sensitive adhesive tape 72 protrudes from both of the end parts of the horizontal line portion 71B in a case where the covering portion 71 is brought into the unfolded state.

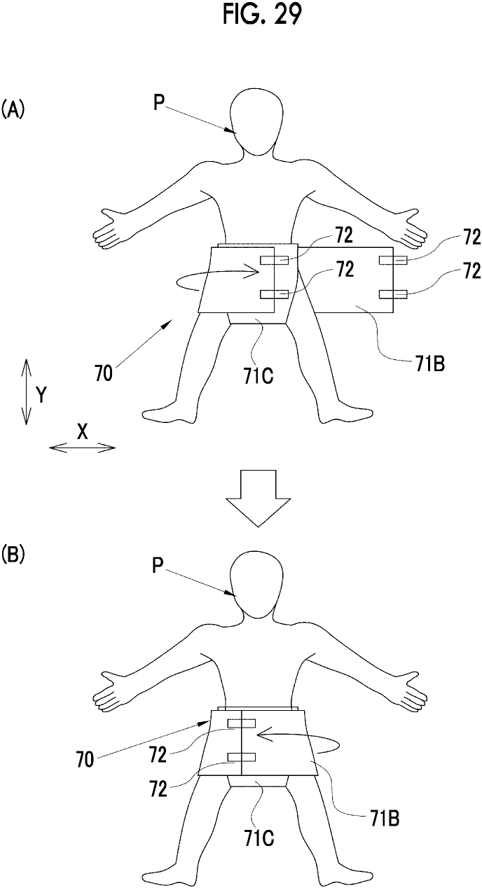

With reference to FIGS. 28 and 29, a method of having the subject P wear the examination garment 70, specifically, a method of wrapping the covering portion 71 around the subject P and a method of causing the pressure-sensitive adhesive tape 72 to adhere, will be described. As shown in (A) of FIG. 28, the doctor D or the helper disposes the covering portion 71 of the examination garment 70 on the rear surface side of the subject P. In this case, it is preferable that the horizontal line portion 71B is disposed parallel to the left-right direction X of the subject P, the vertical line portion 71C is disposed parallel to the up-down direction Y of the subject P, and the first fluid suppression member 22 is aligned with the position of the anus AH. Next, as shown in (B) of FIG. 28, the vertical line portion 71C is wrapped around the torso portion from the thigh portion of the subject P through the vertical line portion 71C between the legs of the subj ect P.

After the vertical line portion 71C is wrapped, as shown in (A) of FIG. 29, the covering portion 71 is wrapped around the torso portion and the thigh portion of the subject P from one end part of the horizontal line portion 71B with respect to the subject P. A portion of the pressure-sensitive adhesive tape 72 that protrudes from the end part of the horizontal line portion 71B is caused to adhere to the covering portion 71. Next, as shown in (B) of FIG. 29, the covering portion 71 is wrapped around the torso portion and the thigh portion of the subject P from the other end part of the horizontal line portion 71B with respect to the subject P. A portion of the pressure-sensitive adhesive tape 72 that protrudes from the end part of the horizontal line portion 71B is caused to adhere to the covering portion 71. Accordingly, the pressure-sensitive adhesive tape 72 can hold a state in which the covering portion 71 is wrapped around the subject P.

As described above, after having the subject P wear the examination garment 70, similarly to the above third embodiment, the doctor D inserts the insertion part 3 of the endoscope 2 into the body of the subject P, for example, the large intestine, from the anus AH through the first fluid suppression member 22 located at the insertion port 21F. Similarly to the above third embodiment, the first fluid suppression member 22 suppresses the passage of the fluid. Thus, it is possible to prevent the body fluid and the droplets from leaking from the insertion port 21F of the examination garment 70. Moreover, since the vertical line portion 71C of the covering portion 71 is covered from the thigh portion of the subject P to the torso portion thereof, the leakage of the body fluid and the droplets can be suppressed as compared to the above third embodiment.

In addition, also in the third embodiment and the modification example thereof, similarly to the above first and second embodiments, any of the storage portion 36 that stores the body fluid released from the subject P, the absorbent material 37, or the discolored portion may be provided on the examination garment 70. In addition, in this case, it is preferable that the examination garment 70 is disposed closer to the left side in the left-right direction X, similarly to the above first embodiment.

In the above third embodiment and modification examples thereof, the pressure-sensitive adhesive tape 72 is exemplified as the holding member that holds a state in which the covering portion 71 is wrapped around the subject P. However, the present invention is not limited to this, and the holding member may include any one of a locking member, a constraint member, and a covering member. It is preferable that the locking member serving as the holding member has a configuration in which at least a part of the covering portion 71 is locked, for example, by a locking structure consisting of a locking claw and a locking hole, a button and a button hole used for clothes, a fitting structure in which a male button and a female button are fitted to each other, or the like. Additionally, as a constraint member serving as the holding member, a paper tape, a rubber band, or the like that constrains the periphery of the covering portion 71 in a state of being wrapped around the subject P is preferable. Additionally, it is preferable that the covering member serving as the holding member has a bag shape that covers the entire covering portion 71 in a state of being wrapped around the subject P.

In the fluid suppression members 22, 24, and 52 in the above respective embodiments, the first porous member 31 or 54 and the second porous member 32 or 55 are each provided with three slits, but the present invention is not limited to this, and four or more slits may be provided. In addition, even in a case where four or more slits are provided, it is preferable that these slits are disposed at equal angle intervals and are connected to each other at the centers of the first porous member 31 and the second porous member 32.

Additionally, in each of the above embodiments, the second fluid suppression member 24, 60, 65, or the like for suction is disposed closer to the left side of the covering portion 21 or 71 in the left-right direction X. However, the present invention is not limited to this, and the second fluid suppression member may be disposed closer to the right side (for example, a position indicated by a two-dot chain line in FIG. 4). Accordingly, the second fluid suppression member 24, 60, 65, or the like for suction is positioned on the left side of the subject P, that is, on the upper side, in a case where the lateral decubitus posture is taken. Thus, this is effective in a case where a large amount of body fluid is stored inside the covering portion, or in a case where it is difficult to insert the suction tube from the lower side of the subject P.

Additionally, in each of the above embodiments, in a case where an endoscopy is performed, the suction tube 15 is inserted into the inside of the examination garment to suction the body fluid. However, the present invention is not limited to this, and after the endoscopy, the suction tube 15 may be inserted into the second fluid suppression member 24, 60, 65, or the like for suction to perform suction. In addition, in this case, after the endoscopy, the insertion part 3 may be removed from the subject P, the first fluid suppression member 22, 52, 60, 65, and the like, and a tube for injecting a cleaning liquid into the first fluid suppression member may be inserted to bring about a state in which the cleaning liquid (for example, water or the like) may be injected into the inside of the examination garments 10, 70, or the like, and the suction tube 15 may be inserted into the second fluid suppression member 24, 60, 65, or the like for suction to perform suction. Alternatively, the examination garment 10, 70, or the like may be separately provided with an injection port for injecting the cleaning liquid, and the injection port may also be provided with the fluid suppression member as in each of the above embodiments.

Additionally, in each of the above-described embodiments, an example in which the covering portion 21 or 71 is formed of a transparent or translucent vinyl sheet is given. However, the present invention is not limited to this, and, for example, the covering portion may be sheets made of polyethylene and polyester other than vinyl or may be formed of paper, cloth, or the like including the material of the diaper. Additionally, instead of making the entire covering portion 21 or 71 transparent or translucent, only the rear surface portion of the covering portion 21 or 71 or a portion near the fluid suppression member may be made transparent or translucent, and the other portions may be made opaque.

EXPLANATION OF REFERENCES

| | |
|---|---|
| 2: | endoscope |
| 3: | insertion part |
| 4: | operating part |
| 5: | universal cord |
| 5A: | connector |
| 10: | examination garment for endoscopy |
| 10A: | front surface portion |
| 10B: | rear surface portion |
| 11: | processor device |
| 12: | light source device |
| 13: | display |
| 14: | user interface (UI) |
| 15: | suction tube |
| 16: | suction device |
| 21: | covering portion |
| 21A: | torso hole |
| 21B: | thigh hole |
| 21C: | string passing portion |
| 21D: | opening portion |
| 21E: | gathering |
| 21F: | insertion port |
| 21G: | fitting hole |
| 21H: | suction tube port |
| 22: | first fluid suppression member |
| 23: | first fixing member |
| 24: | second fluid suppression member |
| 25: | second fixing member |
| 26: | string |
| 27: | string fastener |
| 31: | first porous member |
| 31A to 31C: | first slits |
| 31G: | gap |
| 32: | second porous member |
| 32A to 32C: | second slits |
| 32G: | gap |
| 33: | first frame member |
| 33A: | through-hole |
| 33B: | opening portion |
| 33C: | fitting pin |
| 34: | second frame member |
| 34A: | through-hole |
| 34B: | opening portion |
| 34C: | fitting hole |
| 36: | storage portion |
| 37: | absorbent material |
| 50: | examination garment |
| 52: | first fluid suppression member |
| 53: | first fixing member |
| 54: | first porous member |
| 54A to 54C: | first slits |
| 55: | second porous member |
| 55A to 55C: | second slits |
| 56: | first frame member |
| 56A: | through-hole |
| 56B: | opening portion |
| 56C: | fitting pin |
| 56D: | convex portion |
| 57: | second frame member |
| 57A: | through-hole |
| 57B: | opening portion |
| 57C: | fitting hole |
| 58: | tubular member |
| 58A: | pipe line |
| 59: | fragile portion |
| 60: | fluid suppression member |
| 61: | first porous member |
| 61A: | first slit |
| 62: | second porous member |
| 62A: | second slit |
| 65: | fluid suppression member |
| 66: | duckbill valve |
| 66A: | lip portion |
| 67: | thin plate member |
| 67A: | through-hole |
| 68: | first frame member |
| 68A: | through-hole |
| 68B: | opening portion |
| 69: | second frame member |
| 69A: | through-hole |
| 69B: | opening portion |
| 70: | examination garment |
| 71: | covering portion |
| 71A: | gathering |
| 71B: | horizontal line portion |
| 71C: | vertical line portion |
| 72: | pressure-sensitive adhesive tape |
| AH: | anus |
| CL0: | center line |
| CL1: | center line |
| CL2: | center line |
| D: | doctor |
| F: | body fluid |
| P: | subject |
| T: | examination table |
| X: | left-right direction |
| Y: | up-down direction |
| Z: | insertion direction |

What is claimed is:

1. An examination garment for endoscopy, comprising:
a covering portion that covers a subject and that has an insertion port into which an insertion part of an endoscope is inserted; and
a first fluid suppression member that is provided in the insertion port and that suppresses passage of a fluid in the insertion port.

2. The examination garment for endoscopy according to claim 1,
wherein the covering portion has a suction tube port into which a suction tube is inserted.

3. The examination garment for endoscopy according to claim 2, further comprising:
a second fluid suppression member that is provided in the suction tube port and that suppresses the passage of the fluid in the suction tube port.

4. The examination garment for endoscopy according to claim 1,
wherein the covering portion is a pants-type covering portion including a torso hole through which a torso portion of the subject passes and a thigh hole through which a thigh portion passes.

5. The examination garment for endoscopy according to claim 4,
wherein the covering portion has a contractible member, which contracts the thigh hole, provided around the thigh hole.

6. The examination garment for endoscopy according to claim 5,
wherein the contractible member is a string or an elastic member that is doubly wrapped around the thigh hole.

7. The examination garment for endoscopy according to claim 5,
wherein the contractible member is a gathering that is disposed around the thigh hole.

8. The examination garment for endoscopy according to claim 1,
wherein the covering portion is formed of a planar sheet capable of being wrapped around the subject.

9. The examination garment for endoscopy according to claim 8,
wherein the sheet has a quadrangular outer shape in a case where the sheet is brought into an unfolded state.

10. The examination garment for endoscopy according to claim 8,
wherein the sheet has a T-shaped outer shape in a case where the sheet is brought into an unfolded state.

11. The examination garment for endoscopy according to claim 8, further comprising:
a holding member that holds a state in which the covering portion is wrapped around the subject.

12. The examination garment for endoscopy according to claim 3, further comprising:
a fixing member that attaches at least one of the first or second fluid suppression member to the covering portion.

13. The examination garment for endoscopy according to claim 3,
wherein at least one of the first or second fluid suppression member has at least a first porous member in which a first slit parallel to an insertion direction of the insertion part is formed.

14. The examination garment for endoscopy according to claim 13, further comprising:
a second porous member in which a second slit parallel to the insertion direction and intersecting the first slit is formed.

15. The examination garment for endoscopy according to claim 3,
wherein at least one of the first or second fluid suppression member has a first porous member in which three first slits are formed, and a second porous member in which three second slits are formed.

16. The examination garment for endoscopy according to claim 15,
wherein the first porous member and the second porous member each have a rectangular outer shape,
one of the first slits is disposed to be perpendicular to one side of the first porous member,
one of the second slits is disposed to be perpendicular to one side of the second porous member, and
the first porous member and the second porous member are attached to the covering portion in a state in which the second slit is disposed at a position rotated by 180° around a central axis parallel to the insertion direction with respect to the first slit.

17. The examination garment for endoscopy according to claim 12,
wherein at least one of the first or second fluid suppression member is attached to the covering portion in a state in which the first porous member and the second porous member are laminated.

18. The examination garment for endoscopy according to claim 3,
wherein at least one of the first or second fluid suppression member has
a duckbill valve that maintains airtightness of a pipe line in a state in which the insertion part is not inserted, and
a thin plate member that has a through-hole with an inner diameter that matches an outer diameter of the insertion part and maintains the airtightness of the pipe line in a state in which the insertion part is inserted, and
is attached to the covering portion in a state in which the duckbill valve and the thin plate member are laminated.

19. The examination garment for endoscopy according to claim 12,
wherein the fixing member includes a tubular member that extends to an outside of the covering portion in a case where the covering portion has covered the subject.

20. The examination garment for endoscopy according to claim 19,
wherein at least one of the first or second fluid suppression member has a convex portion that protrudes to the outside of the covering portion in a case where the covering portion has covered the subject.

21. The examination garment for endoscopy according to claim 1,
wherein the covering portion is formed of a transparent or translucent member.

22. The examination garment for endoscopy according to claim 1,
wherein an absorbent material that absorbs a liquid is provided inside the covering portion.

23. The examination garment for endoscopy according to claim 22,
wherein the absorbent material is attachable and detachable with respect to the covering portion.

24. The examination garment for endoscopy according to claim 22,
wherein the absorbent material is provided with a discolored portion of which color changes by absorbing the liquid.

25. The examination garment for endoscopy according to claim 1,
wherein in a case where the covering portion has covered a periphery of a torso hole or the subject, a gathering is provided at a point located on a torso portion of the subject.

26. The examination garment for endoscopy according to claim 4,
wherein a side surface portion and a thigh portion of the covering portion are provided with a fragile portion that is separable with a user’s force.

27. The examination garment for endoscopy according to claim 1,
wherein the covering portion has a cleaning liquid injection port for injecting a cleaning liquid thereinto.

28. The examination garment for endoscopy according to claim 1,
wherein the covering portion has a storage portion that stores a liquid.
